(19) United States
(12) Patent Application Publication
(10) Pub. No.: US 2025/0366708 A1
(43) Pub. Date: Dec. 4, 2025

(54) Systems, Methods, And Apparatus For Attaching An Otoscope Using An Angled Clip-On Device

(71) Applicant: Kenvue Brands LLC, Summit, NJ (US)

(72) Inventors: Lawrence Gerard Ahearn, Branchburg, NJ (US); Meenakshi Lalit Patel, Voorhees, NJ (US); Stephen Anton, Union, NJ (US); Wajeeh Waheed Chaudhry, Birmingham (GB); Aron Baillie Forder, Cambridge (GB); Mark Parry, Cambridge (GB); Sean James Coyle, Pennington, NJ (US)

(21) Appl. No.: 18/733,498

(22) Filed: Jun. 4, 2024

(51) Int. Cl. A61B 1/227; A61B 1/00

(52) U.S. Cl. CPC A61B 1/227; A61B 1/0014

(57) ABSTRACT

A device designed for the attachment of a movable optical element to a smart device may be provided. The device may comprise a main body. The main body may comprise a first member attached to a second member and a third member. An inset angle may be formed between the second and third members. The third member may comprise an aperture. The second member may comprise a first contact surface that may be configured to make contact with a first surface of a smart device. A threaded knob may be connected to a piston through the aperture. The threaded knob may be arranged or configured to move the piston in a direction relative to the first surface when it is turned.

### BACKGROUND

An otoscope may be used for diagnosing and identifying problems of the outer ear and/or middle ear, such as issues with a tympanic membrane, or an ear infection (e.g., acute otitis media). When joined with a camera, the otoscope may be used for remote diagnosis when images or videos may be recorded by the user. A clip device may be used to attach an otoscope to a smart device. However the smart devices may come in various formats, and the clip device may be configured so that it may be compatible with these various formats.

### SUMMARY

Disclosed herein are systems, methods, and apparatus, for attaching a removable optical element, such as an otoscope, to a smart device, such as a smartphone.

In an example, an otoscope clip device may comprise a clip assembly designed to be compatible with one or more smart device sizes. For example, the clip assembly may accommodate the thickness of a variety of smart devices, which may improve compatibility and ensure stability across a diverse array of smart device sizes. Such versatility in the design of the clip assembly may enhance the otoscope clip device's usability in medical settings and may lead to more precise medical examinations and accurate diagnoses.

In an example, the clip assembly may comprise a geometry to minimize the deflection or tilt of the clip assembly when it is attached to a smartphone. The deflection or tilt may refer to the unintended angling or outward bending of the clip assembly (e.g., the otoscope clip device) when it is attached to a smartphone. Deflection or title may cause the otoscope clip device to lose its intended orientation or position. By minimizing the deflection or tilt of the clip assembly, the otoscope clip device may improve the alignment of the smartphone with the otoscope and may improve visibility.

A device designed for the attachment of a movable optical element to a smart device may be provided. The device may comprise a main body. The main body may comprise a first member attached to a second member and a third member. An inset angle may be formed between the second and third members. The third member may comprise an aperture. The second member may comprise a first contact surface. The first contact surface may be configured to make contact with a first smart device surface. The device may comprise a piston, which may comprise a second contact surface. The second contact surface may be configured to come into contact with a second smart device surface. A threaded knob may be connected to the piston through the aperture. The threaded knob may be arranged or configured to move the piston in a direction relative to the first surface when it is turned.

A device may be provided for attaching a moveable optical element to a smart device. The device may comprise a first member. The first member may be referred to as a top member. The first member may comprise a first end and a second end. The device may comprise a second member. The second member may be referred to as a device-side member. The second member may comprise a proximal end that may be attached to the first end of the first member to form a first angle. The first angle may be referred to as a device-side angle. The device may comprise a third member. The third member may be referred to as a dial-side member. The third member may comprise a first aperture and a proximal end. The proximal end of the third member may be attached to the second end of the first member to form a second angle. The second angle may be referred to as a dial-side angle. The device may comprise a piston. The piston may comprise a shaft, a first surface, and a second surface. The first surface may be configured to contact a smart device surface. The second surface may comprise a second aperture for aligning a viewing portion of the moveable optical element with a camera lens of the smart device in (e.g., along) a first axis. The device may comprise a knob connected to the shaft through the first aperture.

A device for attaching to a smart device may be provided. The device may comprise a first member, which may be referred to as a top member. The device may comprise a second member, which may be referred to as a device-side member. The second member may comprise an engagement member. The engagement member may be configured to engage with a movable otoscope in a first position or a second position. The second member may be attached to the first member to form a first angle. The first angle may be referred to as a device-side angle. The device may comprise a third member. The third member may be referred to as a dial-side member. The third member may comprise an aperture. The third member may be attached to the first member to form a second angle, which may be referred to as a dial side angle. The device may comprise a piston. The piston may comprise a shaft and a contact surface. The contact surface may be configured to contact a smart device surface. The device may comprise a knob. The knob may be referred to as a dial. The knob may be connected to the shaft through the aperture.

### BRIEF DESCRIPTION OF THE DRAWINGS

FIGS. 1A-B depict perspective views of an otoscope clip device that is detached from a smart device, such as a smartphone.

FIGS. 1C-D depict perspective views of an otoscope clip device that is attached to a smart device, such as a smartphone.

FIG. 2 depicts a perspective view of an otoscope clip device that comprises an alignment tab and a smart device that may comprise a screen that displays an alignment image.

FIGS. 3A-B depict perspective views of an otoscope clip device that may comprise an otoscope assembly and a clip assembly.

FIGS. 4A-C depict perspective views of an example otoscope clip device where the otoscope assembly is in a first position, which may be an upper position, within a clip assembly.

FIGS. 5A-C depict perspective views of an example otoscope clip device where the otoscope assembly is in a second position, which may be a lower position, within a clip assembly.

FIG. 6 depicts a schematic view of a clip assembly that may comprise one or more components.

FIG. 7 depicts a perspective view of a clip assembly that may comprise a screw clamp assembly.

FIG. 8 depicts a cross-section view of a clip assembly that may comprise a screw clamp assembly.

FIGS. 9A-B depict schematic views of an otoscope assembly that comprises one or more components.

FIG. 10 depicts a side profile view of an angled clip assembly that comprises one or more components.

FIG. 11 depicts a side profile view of a first angled clip assembly that comprises a first geometry, and a second angled clip assembly that comprises a second geometry.

FIG. 12 depicts a side profile view of a first angled clip assembly under a load, and a second angled clip assembly under a load.

FIG. 13 depicts a side profile view of an angled clip assembly that comprises a geometry with a device side angle and a dial side angle.

FIG. 14 depicts a side profile view of an angled clip assembly that comprises a top member that may reduce tilt and/or deflection deformation.

FIG. 15 depicts a side profile view of an angled clip assembly that comprises an internal angle that may reduce tilt and/or deflection deformation.

### DETAILED DESCRIPTION

As remote medicine (e.g., tele-medicine) becomes more common, devices may allow individuals to perform tasks to assist doctors, nurses, or other medical practitioners. For example, a user may take a picture or a video within the ear canal of a patient (e.g., an image of the tympanic membrane), so that a remote doctor may diagnose an issue with the outer ear of the patient. To assist in taking a picture or a video of the outer ear, a user may use an otoscope. For example, a user may connect an otoscope to a camera.

Disclosed herein are systems, methods, and apparatus, for attaching an otoscope to a smart device, such as a smartphone. A user may be provided with the ability to align a camera of the smart device with the otoscope in such a way that a picture or a video (e.g., a clear picture or a clear video) may be taken.

A clip may be provided that may be attached to an otoscope to form an otoscope clip device. The otoscope clip may include a screw clamp assembly (e.g., a clamp), which may open and close using a screw mechanism. The screw clamp assembly may allow a user to align a viewing portion of the otoscope with a camera of a smart device. The screw clamp assembly may allow a user to tune (e.g., finely tune) the alignment of the otoscope clip such that the viewing portion of the otoscope may be aligned with the camera of the smart device.

The clip may include an alignment tab, which may be used to align the viewing portion of the otoscope with the camera of the smart device. For example, an alignment image may be presented on the smart device that may cause the viewing portion of the otoscope to be aligned with the camera of the smart device when the alignment tab is placed over and/or aligned with the alignment image. The alignment image may be an on-screen alignment image, an indicator, a marker, a combination thereof, and/or the like. The alignment image may be an outline of the alignment tab or other shape that may assist in the alignment of the viewing portion of the otoscope with the camera of the smart device.

A user may attach the otoscope clip to the smart device, may align a portion of the otoscope with the camera of the smart device using the alignment tab, and may secure the otoscope clip to the smart device using the screw clamp assembly to close the clamp. The user may then record an image of the outer ear of a patient and may provide that image to a physician for diagnosis. The recorded image may be analyzed by software on the smart device that may work in conjunction with the otoscope clip to provide image analysis, medical analysis, diagnosis, image-taking guidance, and/or the like.

A device may be provided for attaching a movable optical element, such as an otoscope, to a smartphone. The device may comprise a main body. The main body may comprise an aperture and a first engagement member that may be configured to engage with a second engagement structure that belongs to a movable optical element. The movable optical element may be an otoscope. The device may comprise a first surface connected to the main body. The first surface may be configured to contact a first smartphone surface. The first smartphone surface may be a back side of the smartphone, which may comprise a camera. The device may comprise a piston. The piston may comprise a shaft with a threaded hole and a keyway. The piston may comprise a second surface that may be parallel to the first surface and may be configured to contact a second smartphone surface. The second smartphone surface may be a front side of the smartphone that may comprise a smartphone screen. The device may comprise a threaded knob that may be connected to the piston through the aperture. The threaded knob may comprise a reverse thread. The threaded knob may be configured to move the piston in a direction relative to the first surface when turned.

The first surface may be a first non-scratch surface. The second surface may be a second non-scratch surface. A non-scratch surface may be comprised of materials that prevent scratching or damaging another surface, such as a display screen. The materials may be rubber, plastic, fabric, laminate, and/or the like. The first surface and/or the second surface may be or may comprise a gasket. The gasket may prevent slippage, may protect a phone surface, may act as a light block to prevent light from leaking from the otoscope clip device, may prevent light from entering the otoscope clip device optics, and/or may act as a light block to prevent light from entering a camera of the smartphone.

The main body of the device may comprise one or more apertures. For example, the main body may comprise a first aperture that may be a hub that comprises a key that may be complementary to a keyway belonging to a shaft of the piston. The main body may comprise a second aperture that may be configured to be aligned with a viewing portion of the movable optical element.

The piston may further comprise a third surface with an outer edge for aligning a viewing portion of the removable optical element with a camera lens of the smartphone. For example, the outer edge of the third surface may be designed such that its shape may assist a user in aligning the optical element with the camera lens of the smartphone.

A device may be provided for attaching a moveable optical element to a smartphone. The device may comprise a main body. The main body may comprise an aperture and a first engagement member. The first engagement member may be configured to engage with a second engagement member in a first position or a second position. The second engagement member may belong to a movable optical element. The device may comprise a first surface that may be connected to the main body and may be configured to contact a first smartphone surface. The first smartphone surface may be the back side of the smartphone where a camera of the smartphone may be located. The device may comprise a piston. The piston may comprise a shaft, a second surface, and a third surface. The second surface may be parallel or almost parallel to the first surface and may be configured to contact a second smartphone surface. The second smartphone surface may be a display of the smartphone. The third surface may comprise a protrusion for aligning a viewing portion of the moveable optical element with a camera lens of the smartphone in (e.g., along) a first axis. The protrusion may be a protrusion that may correspond to a detent (e.g., an indentation) to assist in securing an alignment.

The device may comprise a knob connected to the shaft through the aperture. The threaded knob may be connected to the piston through the aperture. The threaded knob may be configured to move the piston in a direction relative to the first surface when turned. For example, turning the threaded knob may cause the piston to move the first surface towards the second surface such that the first surface and the second surface may contact the smartphone. This may allow the device to clamp onto the smartphone.

The moveable optical element may be an otoscope. The main body comprises a top portion and a bottom portion. The first engagement member may be connected to the main body and may be configured to engage with the second engagement member in a first position or a second position. The second engagement member may belong to the movable optical element. The first position may be a position within the top portion.

The second position may be a position within the bottom portion. For example, the movable optical element may be moved to the first position, which may be the top portion of the main body. As another example, the movable optical element may be moved to the second position, which may be the bottom portion of the main body. This adjustment may be made to align the smartphone's camera with the movable optical element's viewing section, and/or to accommodate various phone models that may have cameras positioned in different locations.

The third surface of the piston may allow a user to align a viewing portion of the movable optical element with the camera of the smartphone. The third surface may comprise one or more physical features to assist in aligning the viewing portion of the movable optical element with the camera of the smartphone. The third surface may comprise an alignment aperture for aligning the viewing portion of the moveable optical element with the camera lens in (e.g., along) a second axis. The third surface may comprise a color that may match an alignment image that is to be displayed on the smartphone. The third surface may comprise an outer edge that complements an alignment image to be displayed on the smartphone.

A device may be provided for attaching an otoscope to a smartphone. The device may comprise a main body. The main body may comprise an aperture and an engagement member configured to engage with a moveable otoscope in a first position or a second position. The device may comprise a first surface that may be connected to the main body and may be configured to contact a first smartphone surface. The first smartphone surface may be the back side of the smartphone which may be where a camera of the smartphone may be located. The device may comprise a piston. The piston may comprise a shaft and a second surface. The second surface may be parallel to the first surface and may be configured to contact a second smartphone surface. The second smartphone surface may be a display of the smartphone. The device may comprise a threaded knob connected to the shaft through the aperture. The threaded knob may be configured to move the piston in a direction relative to the first surface when turned. For example, when the knob is turned, the first surface may travel towards the second surface such that the first surface and the second surface may contact the smartphone and may allow the device to clamp onto the smartphone.

The otoscope may comprise a removable speculum. The otoscope may comprise a light. The light may be an incandescent light, a light-emitting diode (LED), and/or the like. The light may be powered by a battery, such as a lithium-ion battery, an alkaline battery, and/or the like.

The main body of the device may comprise one or more apertures. For example, the main body may comprise a first aperture that may be a hub and that may comprise a key that is complementary to a keyway that belongs to a shaft of the piston. The shaft may comprise a threaded hole to allow the knob, which may comprise a threaded protrusion, to be connected to the shaft of the piston. In an example, the main body may comprise a second aperture that may be configured to be aligned with a viewing portion of the movable otoscope in a first position or a second position.

A method may be provided for attaching an otoscope clip device to a smart device. A smart device may be placed between a first surface of the otoscope clip device and a second surface of the otoscope clip device. The otoscope clip device may comprise an otoscope. An alignment tab of the otoscope clip device may be aligned with an alignment image that may be on a display of the smart device. A threaded knob of the otoscope clip device may be rotated to cause the first surface of the otoscope clip device to contact the display and the second surface of the otoscope clip device to contact a back surface of the smart device. The back surface of the smart device may comprise a camera of the smart device. A video and/or image of the view through the otoscope of the otoscope clip device may be viewed on the display. An alignment of the otoscope with a camera of the smart device may be adjusted. A video and/or image of a view through the otoscope of the otoscope clip device may be recorded.

Aligning the alignment tab of the otoscope device with the alignment image of the display of the smart device may align the otoscope with the camera of a smart device. Aligning the alignment tab of the otoscope device with the alignment image of the display of the smart device may comprise a one or more actions. A first alignment tab feature of the alignment tab may be aligned with a first object within the alignment image on the display of the smart device to vertically align the otoscope with the camera. A second alignment tab feature of the alignment tab may be aligned with a second object within the alignment image on the display of the smart device to horizontally align the otoscope with the camera. An outer edge of the alignment tab may be aligned with an outline of the alignment image. At least one of the first alignment tab feature and the second alignment tab feature may comprise at least one of a protrusion, a hole, an aperture, a color, a design, an icon, an etching, a symbol, a window, a clear portion of the alignment tab, a combination thereof, or the like.

The otoscope of the otoscope clip device may be moved into a first position or a second position within the otoscope clip device to assist with aligning the otoscope with a camera of the smart device. The otoscope of the otoscope clip device may be moved into a lower position within the otoscope clip device to assist with aligning the otoscope with a camera of the smart device. The otoscope of the otoscope clip device may be moved into an upper position within the otoscope clip device to assist with aligning the otoscope with a camera of the smart device.

A smart device may be used to provide a camera for an otoscope. The smart device may be a smartphone, a smart tablet (e.g., an iPad), a computer, and/or the like. The smart device may include a camera, which may be used by the otoscope to take an image. The camera on the smartphone may provide a cost-effective method of providing the otoscope with the camera. For example, smartphones may now be widely available, and users may already have access to one. By providing a user with a device, such as an otoscope clip device, to attach an otoscope to the smartphone, the user may be given the ability to use the otoscope to take images which may then be sent to a medical professional.

When attaching an otoscope to a smartphone with the otoscope clip, the user may wish to align the otoscope with the camera on the smartphone. Aligning the otoscope with the smartphone camera may provide an image that may not be impeded by a component of the otoscope. Aligning the otoscope with the smartphone camera may help keep stray light out of the image (e.g., seal light out of the otoscope) to improve image quality. To align the otoscope with the camera on the smartphone, a user may have to flip the phone back and forth as the smartphone camera may be on the opposite side of the display.

To minimize flipping the phone back and forth during a camera alignment, an overlay or image may be projected onto the screen of the smartphone. The overlay may be referred to as an alignment image. The alignment image may be complementary to an alignment feature, such as an alignment tab, on the otoscope clip such that a user may align the alignment image with the alignment tab. When the alignment image is aligned with the alignment tab, the viewing portion of the otoscope may be aligned with the smartphone camera. Aligning the alignment image with the alignment tab may allow the user to align the otoscope with the smartphone camera while minimizing turning the phone back and forth during the alignment.

The alignment image may encourage the user to correctly attach the otoscope to the smartphone using the otoscope clip device. For example, the alignment image may be placed at the top of the smartphone to encourage the user to attach the otoscope. The alignment image may be placed at a position on the smartphone to encourage the user to place the otoscope clip over the top of the smartphone. The alignment image may be displayed in such a way as to encourage the user to attach the otoscope clip so that the otoscope clip may not impede one or more buttons of the smartphone when the otoscope clip is attached to the smartphone.

The otoscope clip device may be designed to provide better ergonomics for a user. For example, the otoscope clip device may be designed to improve how a user may be holding a smart device. The otoscope clip may be designed to avoid impeding the ability of the user to place the otoscope in the ear of a patient. The otoscope clip may be designed to encourage a user to correctly place the otoscope in the ear of the patient. For example, the clip may be designed to allow a user to rest their hand against the head of a patient while using the otoscope attached to the smartphone via the otoscope clip.

The otoscope clip may be designed to provide a secure method of attachment to the smart device and may be designed to be attached to one or more styles of smartphones. The otoscope clip may comprise a clamp assembly that may be used to secure the otoscope clip to the smartphone. The clamp assembly may assist in minimizing the movement of the otoscope during the process of taking an image. For example, the clamp assembly may minimize the movement of the clip along an axis of the surface of the smartphone to improve the ability of a user to align the viewing portion of the otoscope with the camera of the smartphone.

The otoscope clip may be designed to avoid compressing one or more buttons of the smartphone. The otoscope clip may be designed to prevent a user from accidentally pressing one or more buttons on the smartphone. For example, the clip may avoid compressing the on/off switch, may avoid compressing a volume switch, may prevent a user from pressing the on/off switch, may prevent a user from pressing a volume switch, a combination thereof, and the like.

A device designed for the attachment of a movable optical element to a smart device may be provided. The device may comprise a main body. The main body may comprise a first member attached to a second member and a third member. An inset angle may be formed between the second and third members. The third member may comprise an aperture. The second member may comprise a first contact surface. The first contact surface may be configured to make contact with a first smart device surface. The device may comprise a piston, which may comprise a second contact surface. The second contact surface may be configured to come into contact with a second smart device surface. A threaded knob may be connected to the piston through the aperture. The threaded knob may be arranged or configured to move the piston in a direction relative to the first surface when it is turned.

In an example, the inset angle formed between the second and third members of the device's main body may be set to range between −2 to 2 degrees. In an example, the range of the inset angle may be adapted to compensate for deflection (e.g., any deflection) the second member may experience under the load imparted by the first smart device surface, which may be a camera surface. In an example, the inset angle may be adapted to counteract the deflection caused by the load from the camera surface.

The main body of the device may be composed of a resilient material, which may allow the inset angle to change (e.g., increase) when a load is applied to the first contact surface. The main body may comprise materials such as Polycarbonate-Acrylonitrile Butadiene Styrene (PC ABS), glass-filled PC ABS (PC ABS G), and the like to provide resilience and functional elasticity.

The structure and/or geometry of the device may be such that adaptability under load may be observed. In an example, the second member may be adapted to bend away from the third member upon application of a first load to the first contact surface. In an example, when a second load is applied to the second contact surface, the third member may be adapted to bend away from the second member. This may be done, for example, to ensure balance and flexibility in the device's operation.

In an example, the first member of the main body may comprise a thickness ranging between 3 mm to 6 mm. The thickness may aid in reducing flex at the junctions where the first member may meet the second and third members. This may, for example, enhance the structural integrity of the device and/or prevent deflection that may be caused by a surface of the smart device (e.g., a camera surface).

In an example, attachment of the first member to both the second and third members may involve a first end of the first member being connected to a proximal end of the second member to form a first angle and a second end of the first member being connected to a proximal end of the third member to create a second angle. The angles formed between the members may contribute to the overall geometric stability and functionality of the device and/or may prevent deflection caused by a surface of the smart device (e.g., a camera surface). For example, the first angle may be between 70 to 100 degrees, and the second angle may be between 80 to 110 degrees.

A device may be provided for attaching a moveable optical element to a smart device. The device may comprise a first member. The first member may be referred to as a top member. The first member may comprise a first end and a second end. The device may comprise a second member. The second member may be referred to as a device-side member. The second member may comprise a proximal end that may be attached to the first end of the first member to form a first angle. The first angle may be referred to as a device-side angle. The device may comprise a third member. The third member may be referred to as a dial-side member. The third member may comprise a first aperture and a proximal end. The proximal end of the third member may be attached to the second end of the first member to form a second angle. The second angle may be referred to as a dial-side angle. The device may comprise a piston. The piston may comprise a shaft, a first surface, and a second surface. The first surface may be configured to contact a smart device surface. The second surface may comprise a second aperture for aligning a viewing portion of the moveable optical element with a camera lens of the smart device in (e.g., along) a first axis. The device may comprise a knob connected to the shaft through the first aperture.

In an example, the first angle formed may be within a range between 70 to 100 degrees. The second angle may be within a range between 80 to 110 degrees.

In an example, the device may comprise a third angle that is formed between the second member and the third member.

In an example, the device may further comprise a third angle that is formed between the second member and the third member. The third angle may be between −2 to 2 degrees.

In an example, the first smart device surface may be a camera surface. At least one of the first angle, the second angle, or the third angle may be configured to compensate for a deflection of the second member under a load caused by the camera surface.

In an example, the second member may be configured to bend away from the third member when a first load is applied to the first contact surface.

In an example, the third member may be configured to bend away from the second member when a second load is applied to the second contact surface.

In an example, the first member may comprise a thickness between 3 mm to 6 mm and may be configured to reduce flex where at least one of the proximal end of the second member is attached to the first end, or the proximal end of the third member is attached to the second end.

A device for attaching to a smart device may be provided. The device may comprise a first member, which may be referred to as a top member. The device may comprise a second member, which may be referred to as a device-side member. The second member may comprise an engagement member. The engagement member may be configured to engage with a movable otoscope in a first position or a second position. The second member may be attached to the first member to form a first angle. The first angle may be referred to as a device-side angle. The device may comprise a third member. The third member may be referred to as a dial-side member. The third member may comprise an aperture. The third member may be attached to the first member to form a second angle, which may be referred to as a dial side angle. The device may comprise a piston. The piston may comprise a shaft and a contact surface. The contact surface may be configured to contact a smart device surface. The device may comprise a knob. The knob may be referred to as a dial. The knob may be connected to the shaft through the aperture.

In an example, the first angle may be between 70 to 100 degrees. The second angle may be between 80 to 110 degrees.

In an example, the device may comprise a third angle that is formed between the second member and the third member. The third angle may be between −2 to 2 degrees.

In an example, the smart device surface may be a camera surface. At least one of the first angle, the second angle, or the third angle may be adopted to compensate for a deflection of the second member under a load caused by the camera surface.

In an example, the second member may be configured to bend away from the third member when a first load is applied to the contact surface.

In an example, the third member may be configured to bend away from the second member when a second load is applied to the contact surface.

In an example, the first member may comprise a thickness between 3 mm to 6 mm and may be adapted to reduce flex where at least one of the second member is attached to the first member or the third member is attached to the first member.

In an example, an otoscope clip device may comprise a clip assembly designed to be compatible with one or more smart device sizes. For example, the clip assembly may accommodate the thickness of a variety of smart devices, which may improve compatibility and ensure stability across a diverse array of smart device sizes. Such versatility in the design of the clip assembly may enhance the otoscope clip device's usability in medical settings and may lead to more precise medical examinations and accurate diagnoses.

In an example, the clip assembly may comprise a geometry to minimize the deflection or tilt of the clip assembly when it is attached to a smartphone. The deflection or tilt may refer to the unintended angling or outward bending of the clip assembly (e.g., the otoscope clip device) when it is attached to a smartphone. Deflection or title may cause the otoscope clip device to lose its intended orientation or position. By minimizing the deflection or tilt of the clip assembly, the otoscope clip device may improve the alignment of the smartphone with the otoscope and may improve visibility.

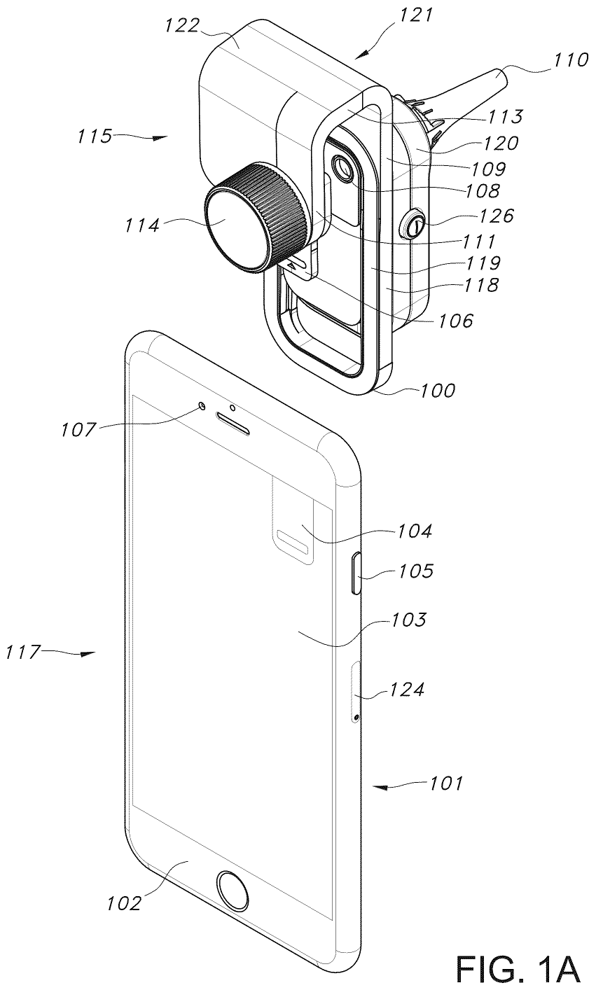

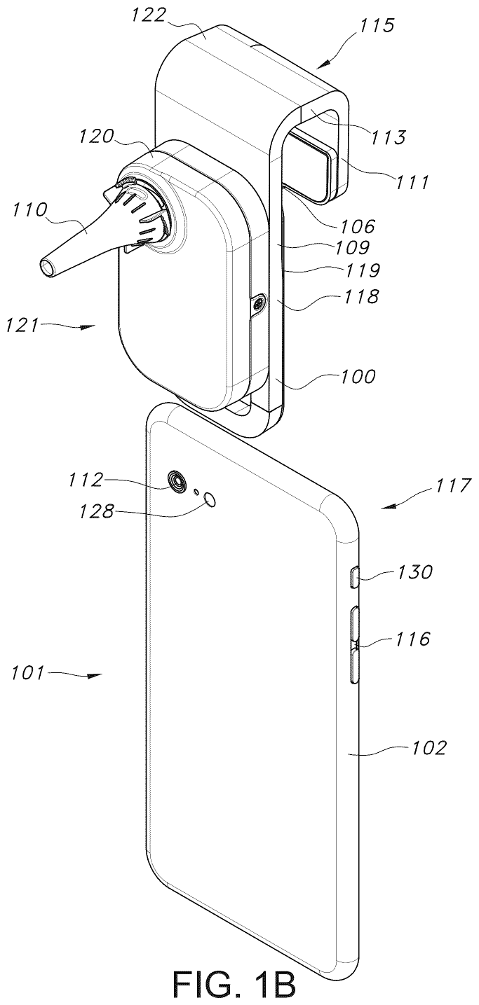

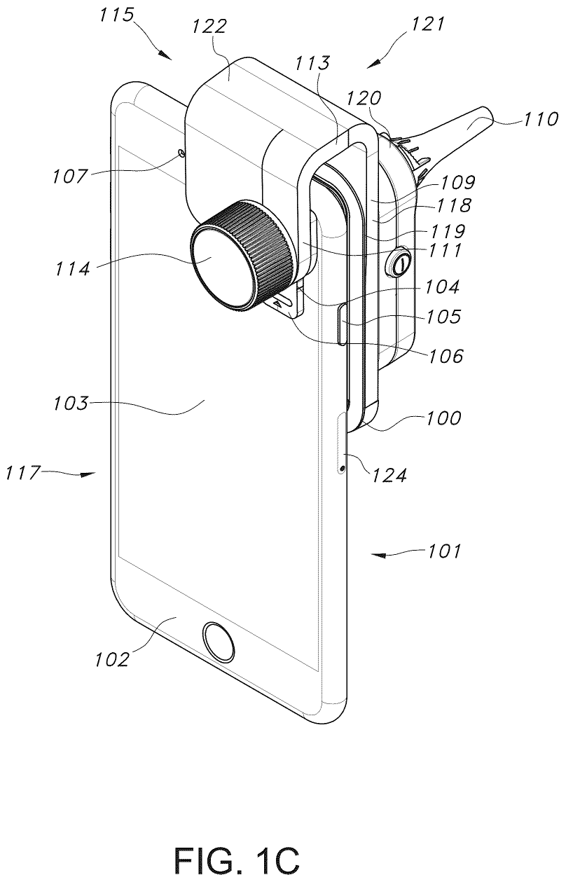

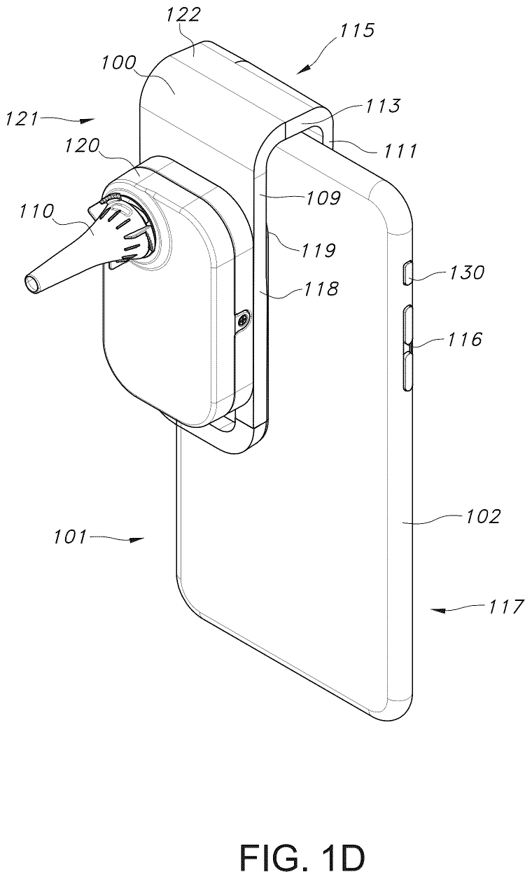

FIGS. 1A-B depict perspective views of an otoscope clip device that is detached from a smart device, such as a smartphone. As shown in FIG. 1A, the otoscope clip device 100 may be detached from the smart device 102. FIG. 1A may depict the front side (e.g., front side 117) of smart device 102, and the back side (e.g., dial-side 115) of the otoscope clip device 100. The back side (e.g., dial-side 115) of the otoscope clip device 100 may comprise a knob (e.g., a dial), such as the knob 114, and an alignment tab, such as the alignment tab 106. The front side (e.g., front side 117) of the smart device may comprise a camera, such as the camera 107, and a display screen, such as the display 103. The camera 107 may be directed towards a user. The front side (e.g., front side 117) of the smart device may comprise at least one of a button, a speaker, a microphone, a light, a combination thereof, and the like. The button may be button 105, which may be the home button. The speaker may allow a user to listen to a phone call. The microphone may allow a user to speak during a phone call. The light may function as a camera flash when the camera is used for taking a photo. The button 105 may be located on the front side (e.g., front side 117) of smart device 102.

The smart device 102 may be a smartphone, a smart tablet (e.g., an iPad), a computer, and the like. The smart device 102 may comprise a display, such as display 103. The display 103 may be a liquid crystal display (LCD) and may be located on the front-facing portion of the smart device (e.g., front side 117). The display 103 may show an alignment image 104. The alignment image 104 may assist the user in aligning a viewing portion of an otoscope (e.g., viewing portion 108) with a camera of the smart device, such as the camera at 112 shown in FIG. 1B.

Referring again to FIG. 1A, the alignment image 104 may be complementary to an alignment feature, such as the alignment tab 106, on the otoscope clip device 100 such that a user may align the alignment image 104 with the alignment tab 106. When the alignment image 104 is aligned with the alignment tab 106, the viewing portion 108 of the otoscope may be aligned with the smart device camera 112. Aligning the alignment image 104 with the alignment tab 106 may allow the user to align the viewing portion 108 of the otoscope with the smart device camera 112 (shown in FIG. 1B) while minimizing turning the phone back and forth during the alignment.

The otoscope clip device 100 may comprise one or more components. For example, the otoscope clip device 100 may comprise the clip assembly 122 and/or the otoscope assembly 120.

The clip assembly 122 may comprise dial-side member 111, device-side member 109, and top member 113. The top member 113, may be attached to both device-side member 109 and the dial-side member 111 to form a main body. In an example, the clip assembly 122 may be a unibody design such that dial-side member 111, device-side member 109, and top member 113 are integrated into the clip assembly 122.

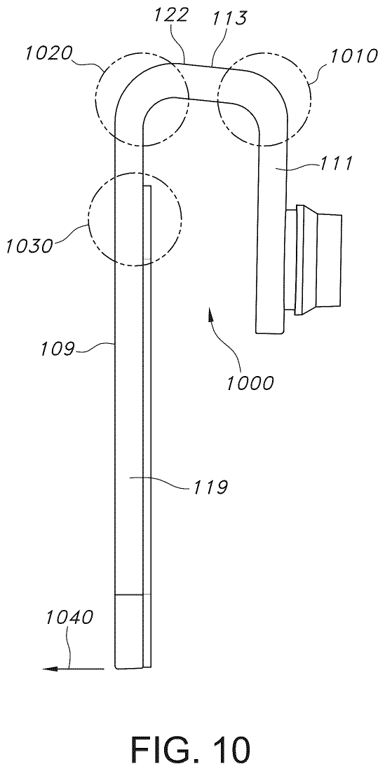

In an example, device-side member 109 may be attached to a first end of top member 113 to form a device-side angle (e.g., 1020 in FIG. 10). The dial-side member 111 may be attached to the second end of top member 113 to form a dial-side angle (e.g., 1010 in FIG. 10). This structural arrangement, which may be referred to as a configuration or a geometry, may ensure the integral assembly of the clip assembly 122 and may allow for the maintenance of a range of angles between its components. For example, the geometry of clip assembly 122 may prevent tilt and deflection deformation caused by a surface of the smart device (e.g., a camera surface) to ensure functionality and effectiveness when attaching a movable optical element to the smart device.

The adaptability and resilience of clip assembly 122 may be such as to accommodate varying designs of smartphone cameras. For example, the device-side member 109, the top member 113, and dial-side member 111 may be designed with flexibility in mind. The materials chosen for these components may possess resilience, allowing for a degree of flexion. This flexibility may allow clip assembly 122 to adapt to smartphones where cameras may protrude from the device's surface, which may be referred to as a camera bump. The contents or members of clip assembly 122 may be constructed in such a manner that they may flex to embrace these variances while minimizing the risk of deflection.

Deflection deformation may refer to the bending or warping of the clip assembly in areas that may be subjected to concentrated pressure, resulting in a member of the clip moving from an intended position. For example, deflection deformation may refer to the undesirable scenario where a portion of clip assembly 122, such as device-side member 109, may lose contact with the surface of the smart device. A distance of deflection, or deflection distance, may indicate how a member of the clip may move from an intended position under stress. Deflection deformation may result in misalignment of the otoscope and the smart device camera as the deformed clip may not be able to hold the otoscope in a correct position relative to the camera, leading to suboptimal image quality or the inability to use the otoscope effectively.

By reducing deflection, the design of clip assembly 122 may ensure an enlarged contact surface area between device-side member 109 or one of its surfaces and the smart device, which may maintain a secure and stable connection. Reducing or preventing deflection may also improve image quality and the ability to use the otoscope effectively.

In an example, the design of clip assembly 122 may ensure that regardless of the presence of a camera bump or the slight variations in the external features of smart devices, the clip assembly maintains optimal contact with the smart device, enhancing the stability and usability of the attachment.

The otoscope clip device 100 may comprise the alignment tab 106. The alignment tab may be a part of the clip assembly 122. The alignment tab 106 may include one or more features to assist in the alignment of the viewing portion 108 of the otoscope. The one or more features may include a protrusion, a hole, an aperture, a color, a design, an icon, an etching (e.g., a laser etching), a symbol, and/or the like.

The alignment tab 106 may include one or more alignment protrusions. An alignment protrusion of the one or more alignment protrusions may be a protrusion on a surface of the alignment tab 106 that may be aligned with a portion of alignment image 104. For example, the alignment tab 106 may include an arrow-shaped alignment protrusion that may be aligned with a line within a portion of the alignment image 104.

The alignment tab 106 may include an alignment design. The alignment design may be an etching on the surface of the alignment tab 106 to assist in the alignment of the viewing portion 108 of the otoscope. For example, the alignment design may be one or more intersecting lines, such as a crosshair, that when aligned with an alignment image on a smart device may align the viewing portion 108 of the otoscope with a camera of the smart device.

The alignment tab 106 may include an outer edge that may be aligned with an outer edge of the alignment image 104. The outer edge of the alignment tab 106 may be a shape that may be complementary to the outer edge of the alignment image 104. When the outer edge of the alignment tab 106 is aligned with the outer edge of the alignment image 104, the viewing portion 108 of the otoscope may be aligned with the camera of the smart device.

The alignment tab 106 may include an aperture that may be aligned with an object within the alignment image 104. For example, the aperture of alignment tab 106 may align with a bullseye target on the alignment image 104, an oval shape within the alignment image 104, a line within the alignment image 104, a combination thereof, and/or the like. The alignment aperture may assist in the alignment of the viewing portion 108 of the otoscope. For example, the alignment aperture may cause the viewing portion 108 of the otoscope to be aligned with the camera of the smart device when the alignment aperture is aligned with a corresponding shape within an image on a display of the smart device.

The alignment tab 106 may be made of a material of a color that may match a color of the alignment image 104. The alignment tab 106 may be made of a material of a color that may be complementary to the color of alignment image 104. The alignment tab 106 may be made of a clear material such that a user may see through the clear material and align the alignment tab 106 with the alignment image 104. The alignment tab 106 may be made of a material, such as a resilient material, which may be able to apply pressure to the surface of the smartphone. For example, alignment tab 106 may be made of plastic, metal, a polymer, and/or the like.

The alignment tab 106 may be placed in a parallel position or a near parallel position to another surface of the otoscope clip device 100, such as a surface of the device-side member 109 and/or the clip engagement member 118. Device-side member 109 may be or may comprise clip engagement member 118. The alignment tab 106 may be placed in a parallel position or almost parallel position to another surface of the otoscope clip device 100 such that the otoscope clip device 100 may form a c-shape. The c-shape that may be formed using the parallel or near parallel position of alignment tab 106 with another surface of the otoscope clip device 100, such as a surface of device-side member 109 and/or clip engagement member 118, and may be used to provide a clamp, such as a c-clamp. The shape of the c-clamp may be formed by the clip assembly 122 (e.g., the dial-side member 111, top member 113, and the device-side member 109). The clamp may be driven by a knob or dial, such as the knob 114, which may be found on the dial-side 115 of the otoscope clip device 100.

The otoscope clip device 100 may comprise knob 114, which may be a knob or dial that may drive a screw clamp assembly. The knob 114 may be a part of the clip assembly 122 and may be found on the dial-side 115 of the clip assembly 122. The knob 114 may be a cylindrical shape with a flat top surface, a bottom surface with at least a hole, and an outer surface. The outer surface of knob 114 may include one or more splines, knurling, or other texture which may provide a gripping surface. When turned, the knob 114 may engage a screw clamp assembly such that a piston that may include alignment tab 106 may be moved toward a surface that is parallel or near parallel to the alignment tab 106. For example, a user may turn the knob 114 so that the alignment tab 106 moves towards a parallel surface of the otoscope clip device 100. The knob 114 may cause the alignment tab 106 to move towards clip engagement member 118 such that alignment tab 106 and the clip engagement member 118 may clamp onto the smart device 102.

The otoscope clip device 100 may comprise clip engagement member 118. The clip engagement member 118 may be part of the clip assembly 122 and/or the device-side member 109. For example, device-side member 109 may comprise clip engagement member 118. The clip engagement member 118 may have a first surface and a second surface. The first surface of the clip engagement member 118 may face the back side (e.g., back side 101 shown in FIG. 1B) of the smart device 102. The first surface of the clip engagement member 118 may include an anti-skid material, and/or an anti-scratch material. For example, the first surface of the clip engagement member 118 may be made of rubber to prevent scratching and/or damage to the smart device 102 and to prevent the otoscope clip device 100 from moving when it is attached to the smart device 102. The clip engagement member 118 may include an elongated aperture. The elongated aperture may allow the viewing portion 108 to protrude into or through the clip engagement member 118. This may allow viewing portion 108 to be moved into a number of positions such that the viewing portion 108 may be aligned with smart device camera 112.

The clip engagement member 118 may include a number of features to allow the viewing portion 108 to be placed in one or more positions. For example, the clip engagement member 118 may include a protrusion in a side of the clip engagement member 118 that may have a complimentary notch (e.g., indentation, or detent) in the otoscope assembly 120, which may allow the otoscope assembly 120 and/or the viewing portion of the otoscope to be locked in a position.

The clip engagement member 118 may allow the otoscope assembly 120 to be attached and/or connected to clip assembly 122. The clip engagement member 118 may be a portion of a c-clamp that is part of the clip assembly 122. The clip engagement member 118 may allow the otoscope assembly 120 to be movably attached to the clip assembly 122. For example, the clip engagement member 118 may allow otoscope assembly 120 to move into one or more positions within the clip assembly 122. The clip engagement member 118 may allow the otoscope assembly 120 to be removably attachable to the clip assembly 122. For example, the clip engagement member 118 may allow otoscope assembly 120 to be attached to and/or removed from the clip assembly 122.

The contact surface 119 may be in contact with the clip engagement member 118. The contact surface 119 may comprise an anti-skid material that may prevent slippage. The contact surface 119 may be or may comprise a gasket. The gasket may prevent slippage, may protect a phone surface, and/or may act as a light block to prevent light leakage from the device. For example, the gasket may prevent ambient light from entering the optics.

The otoscope clip device 100 may comprise a viewing portion of an otoscope, such as the viewing portion 108. The viewing portion of the otoscope may be a part of the otoscope assembly 120. The viewing portion 108 may be where an image may come into focus from the usage of the otoscope such that the image may be viewed at viewing portion 108. The viewing portion 108 of the otoscope may be where a user or a camera may look into the otoscope. For example, the viewing portion 108 may be used to view into an ear of a patient, may be used to take a picture or a video of an outer ear, an ear canal of the patient, and/or the like. As described herein, the viewing portion of 108 may come in contact with a camera or may be aligned with a camera.

The otoscope clip device 100 may comprise an otoscope assembly 120. The otoscope assembly 120 may comprise an otoscope. The otoscope assembly 120 may comprise the outer tip housing 110. The otoscope assembly 120 may comprise the light switch 126. The light switch 126 may control a light that may be within or near the outer tip housing 110. For example, the light switch may turn on or off a light, which may be battery powered, that may be seen through an opening in the distal end of the outer tip housing 110. The light switch 126 may be a switch, a button, and the like. This may be done, for example, to provide light within an outer ear, an ear canal of a patient, and the like such that a photo may be taken.

The otoscope clip device 100 may comprise the outer tip housing 110. The outer tip housing 110 may have a distal end with an opening that may peer into an outer ear and/or an ear canal of a patient when a speculum is attached to the outer tip housing 110. The outer tip housing 110 may have a proximal end that may be connected to the otoscope assembly 120. The outer tip housing 110 may include a radial slot that may allow a removable speculum to be attached to the outer tip housing 110. For example, the removable speculum may be attached to the outer tip housing 110 via the radial slot such that the otoscope assembly 120 may be used to take a picture or a video of an outer ear and/or an ear canal of a patient. The otoscope portion of the otoscope clip device 100 may provide an optic magnification of the outer ear and/or ear canal of the patient.

As shown in FIG. 1B, the otoscope clip device 100 may be detached from the smart device 102. FIG. 1B shows the back side (e.g., back side 101) of the smart device, and a front side of the otoscope clip device 100 (e.g., device-side 121). The back side 101 of the smart device 102 may include the light 128, and a smartphone camera, such as the smart device camera 112. The smart device camera 112 may face away from a user when the user is viewing the display screen, such as the display 103 of the smart device 102.

As described here in the otoscope clip device 100 may include the outer tip housing 110. The outer tip housing 110 may include an opening. A removable speculum may attach to and/or cover the outer tip housing 110. When the alignment tab 106 is aligned with the alignment image 104, the outer tip housing 110 may be situated in such a way as to be aligned with smart device camera 112. For example, an opening of the outer tip housing 110 may be aligned with the smart device camera 112, and the viewing portion 108 of the otoscope may be aligned with the smart device camera 112.

FIGS. 1C-D depict perspective views of an otoscope clip device that is attached to a smart device, such as a smartphone. As shown in FIG. 1C, otoscope clip device 100 may be attached to smart device 102. FIG. 1C may depict the front side of a smart device 102, and the back side of the otoscope clip device 100. The back side of the otoscope clip device 100, which may be referred to as the dial-side 115, may comprise a knob (which may be referred to as a dial), such as the knob 114, an alignment tab, such as the alignment tab 106. The front side of the smart device may comprise a camera, such as the camera 107, which may be directed towards the user, and a display screen, such as display 103.

The otoscope clip device 100 may be attached to the smart device 102 by a clamping mechanism, such as a screw clamp assembly, which may be driven by the knob 114. The knob 114 may cause the screw clamp assembly to close when turned. For example, the knob 114 may cause the screw clamp assembly to close when turned in a clockwise direction. The knob 114 may cause the screw clamp assembly to open when turned. For example, the knob 114 may cause the screw clamp assembly to open when turned in a counterclockwise direction. When the screw clamp assembly is closed, the otoscope clip device 100 may be attached to the smart device 102. When the screw clamp assembly is open, the otoscope clip device 100 may be detached from the smart device 102.

When turned, the knob 114 may engage a screw clamp assembly such that the screw clamp assembly may close. The knob 114 may cause a piston that may include the alignment tab 106 to move towards a surface that is parallel or near parallel to the alignment tab 106. For example, the knob 114 may cause the alignment tab 106 to move towards clip engagement member 118 such that the alignment tab 106 may come in contact with a display of smart device 102, and clip engagement member 118 may come in contact with the back side (e.g., the back side 101) of smart device 102. The alignment tab 106 and/or the engagement member may include a surface that may come in contact with the display 103. The surface of the alignment tab 106 may be made of a material that may be an anti-scratch and/or anti-skid.

The otoscope clip device 100 may be attached to the smart device 102 in such a way as to avoid compressing and/or contacting one or more buttons of the smart device 102. The otoscope clip device 100 may attach to the smart device 102, such that otoscope clip device 100 may contact one or more of a front face (e.g., front side 117), a back face (e.g., back side 101), and a top portion of smart device 102. Otoscope clip device 100, may be attached in such a way as to avoid contacting the sim card cover 124, the button 105, the volume button 116, and the switch 130 (the volume button 116 and the switch 130 may be shown with respect to FIG. 1D) of the smart device 102.

As shown in FIG. 1C, alignment tab 106 may be aligned with alignment image 104. For example, alignment tab 106 may cover alignment image 104. Alignment tab 106 may cover alignment image 104 such that alignment tab 106 may remain within the edges of the alignment image 104.

When alignment tab 106 may be within and/or aligned with the alignment image 104, the otoscope assembly 120 of the otoscope clip device 100 may be aligned with the smart device camera 112.

As shown in FIG. 1D, the otoscope assembly 120, the viewing portion 108 of the otoscope, and/or the outer tip housing 110 may be aligned with the smart device camera 112. The otoscope assembly 120 may be aligned with the smart device camera 112 in such a way that a user may place the otoscope with speculum in the ear of a patient and may take a photo of the outer ear and/or ear canal of the patient. For example, the smart device camera 112 may be able to use the otoscope assembly 120 to view an outer ear and/or an ear canal of a patient via a sight path that may be established through the viewing portion 108 of the otoscope and through an opening in the outer tip housing 110 and through the attached speculum.

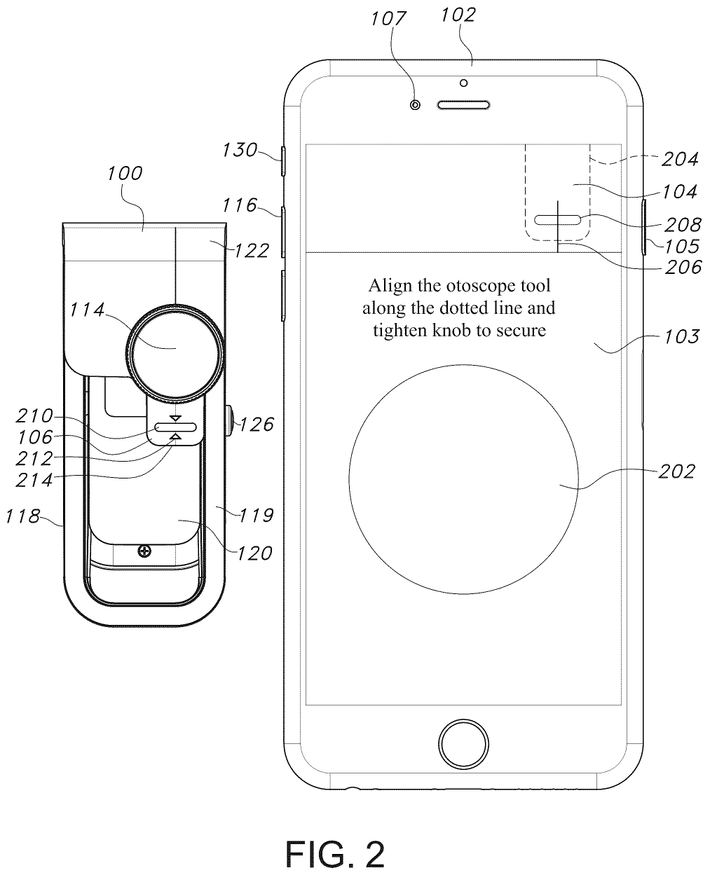

FIG. 2 depicts a perspective view of an otoscope clip device that comprises an alignment tab and a smart device that may comprise a screen that displays and alignment image.

The smart device 102 may comprise a one or more buttons and/or switches. For example, the smart device 102 may comprise the switch 130, the volume buttons 116, and the button 105. The smart device may include a camera, such as the camera 107, which may face a user. The smart device may include a display, such as the display 103.

The display 103 may provide instructions to a user as to how to align the otoscope clip device 100. The display 103 may include the image 202, which may be an image taken from the smart device camera. When otoscope clip device 100 may be attached to the smart device 102 an image may be taken. For example, an image may be taken through an otoscope. For example, the otoscope clip device 100 may include the otoscope assembly 120. When otoscope clip device 100 is attached to the smart device 102, the otoscope assembly 120 may be aligned with a smart device camera and an image or video may be taken through the otoscope associated with the otoscope assembly 120.

The display 103 may display the alignment image 104. The alignment image 104 may be used to assist a user in aligning and otoscope with a smart device camera when the user is attaching the otoscope clip device 100 to the smart device 102. The alignment image 104 may depict a shape that may be similar and/or complementary to the alignment tab 106 of the otoscope clip device 100. When the alignment tab 106 may be placed within the alignment image 104, an otoscope associated with the otoscope assembly 120 may be aligned and/or may be in contact with a smart device camera.

The alignment image 104 may include a number of features that may assist in aligning the alignment image 104 with the alignment tab 106 and/or the otoscope associated with the otoscope assembly 120. These features may include a shape of the alignment image 104, the color of the alignment image 104, a shape within the alignment image 104, an object within the alignment image 104, a color within the alignment image 104, a combination thereof, and/or the like. The alignment image 104 may include the alignment image outline 204, the horizontal alignment image feature 206, the vertical alignment image feature 208, a combination thereof, and/or the like.

The alignment image 104 may be used by a user to determine how to move the otoscope clip device 100 to achieve alignment. The alignment image 104 may be used by the user to determine how to move the otoscope clip device 100 in an axis associated with a plane created by the display 103. For example, the display 103 may have a horizontal axis that may cross a first side of smart device 102 that may include volume button 116 to a second side of smart device 102 that may include button 105. The display 103 may have a vertical axis that may be orthogonal to the horizontal axis. The alignment image 104 may provide a user with visual feedback as to how to move the otoscope clip device 100 in the vertical axis and/or the horizontal axis of the display 103, which may align the otoscope with a smart device camera.

The alignment image outline 204 may allow user to align the alignment tab 106 of the otoscope clip device 100 such that the otoscope associated with the otoscope assembly 120 may be aligned with the smart device camera. The alignment image outline 204 may be used by the user to align the alignment tab 106 in the vertical axis and/or the horizontal axis of the display 103. For example, the user may place the alignment tab 106 on, near, or within the alignment image outline 204 and to achieve alignment. If the otoscope is misaligned with the smart device camera, a portion of the alignment tab 106 may appear outside of the alignment image outline 204. The user may adjust the otoscope clip device 100 to address the portion of the alignment tab 106 that may be outside of the alignment image outline 204 and may align the otoscope with the device camera. By correcting the misalignment of the alignment tab 106 with the alignment image outline 204, the user may correct the alignment of the otoscope with the smart device camera.

The alignment image outline 204 may be larger (e.g., slightly larger) than the alignment tab 106 such that alignment tab 106 may fit within the alignment image 104 and the alignment image outline 204 may be seen. The alignment image outline 204 may be the same or of a similar color to the alignment image 104, the alignment tab 106, and/or the alignment tab edge 214. The alignment image outline 204 may be a different color than the alignment image 104, the alignment tab 106, and/or the alignment tab edge 214.

The horizontal alignment image feature 206 may be used by the user to determine how to move the otoscope clip device 100 to achieve alignment. The horizontal alignment image feature 206 may be a line, an object, a shape, an icon, and indicator, and/or the like. For example, the horizontal alignment image feature 206 may be a line elongated in a vertical direction. The horizontal alignment image feature 206 may be within the alignment image 104, may be the outside alignment image 104, and/or may intersect the alignment image outline 204. The horizontal alignment image feature 206 may be used by a user to determine how to move the otoscope clip device 100 along a horizontal axis associated with the display 103 to horizontally align the otoscope associated with the otoscope assembly 120 with the smart device camera.

To provide guidance as to how to move the otoscope clip device 100 along the horizontal axis, the horizontal alignment image feature 206 may have one or more reference points. For example, the horizontal alignment image feature 206 may be a number of dots along a vertical axis, a line along the vertical axis, a shape elongated along the vertical axis, an indicator along the vertical axis, and/or the like.

To align the otoscope with the smart device camera along the horizontal axis, a user may attach the otoscope clip device 100 to the smart device 102 such that the horizontal alignment tab feature 212 may be aligned with the horizontal alignment image feature 206. For example, the user may align the arrow or triangular protrusions of the horizontal alignment tab feature 212 with the line of the horizontal alignment image feature 206.

The vertical alignment image feature 208 may be used by the user to determine how to move the otoscope clip device 100 to achieve alignment. The vertical alignment image feature 208 may be a line, an object, a shape, an indicator, an icon, and/or the like. For example, the vertical alignment image feature 208 may be an oval elongated along the horizontal axis. The vertical alignment image feature 208 may be within alignment image 104, may be outside alignment image 104, and/or may intersect the alignment image outline 204. The vertical alignment image feature 208 may be used by a user to determine how to move the otoscope clip device 100 along the vertical axis associated with the display 103 to vertically align the otoscope associated with the otoscope assembly 120 with the smart device camera.

To provide guidance as to how to move the otoscope clip device 100 along the vertical axis, the vertical alignment image feature 208 may have one or more reference points. For example, the vertical alignment image feature 208 may be a number of dots along a horizontal axis, a line along the horizontal axis, a shape elongated along the horizontal axis, an indicator along the horizontal axis, and/or the like.

To align the otoscope with the smart device camera along the vertical axis, a user may attach the otoscope clip device 100 on the smart device 102 such that the vertical alignment tab feature 210 may be aligned with the vertical alignment image feature 208. For example, the user may align the oval aperture of the vertical alignment tab feature 210 with the vertical alignment image feature 208.

The otoscope clip device 100 may comprise the clip assembly 122 and the otoscope assembly 120. The otoscope assembly 120 may include an otoscope and may include a light for the otoscope. The otoscope assembly 120 may include the light switch 126 that may control the light for the otoscope. Although a switch is shown with respect to the light switch 126, another suitable element, such as a button, may be used. For example, an On/Off button, a push button, a switch, and/or the like may be used.

The inside portion of the otoscope assembly 120 may be seen in FIG. 2. When the otoscope clip device 100 may be attached to smart device 102, the inside portion of the otoscope assembly 120 may face and/or contact a back portion of the smart device 102 that may include a smart device camera.

The clip assembly 122 may comprise a clamp assembly that may include the knob 114. The clip assembly 122 may comprise the clip engagement member 118. The clip assembly 122 may comprise alignment tab 106.

The alignment tab 106 may include one or more features that may assist in aligning the alignment tab 106 with the alignment image 104 and/or the otoscope associated with the otoscope assembly 120. These features may include a shape of the alignment tab 106, the color of the alignment tab 106, the shape within the alignment tab 106, an object within the alignment tab 106, a color within the alignment tab 106, a combination thereof, and/or the like. The alignment tab 106 may include the alignment tab edge 214, the horizontal alignment tab feature 212, the vertical alignment tab feature 210, a combination thereof, and/or the like.

The alignment tab 106 may be used by a user to determine how to move the otoscope clip device 100 to achieve alignment. The alignment tab 106 may be used by a user to determine how to move the otoscope clip device 100 in an axis associated with a plane created by the display 103. For example, the display 103 may have a horizontal axis that may cross a first side of the smart device 102 that includes the volume button 116 to a second side of the smart device 102 that includes the button 105. The display 103 may have a vertical axis that may be orthogonal to the horizontal axis. The alignment tab 106 may provide a user with feedback as to how to move the otoscope clip device 100 in the vertical axis or the horizontal axis of the display 103, which may align the otoscope with a smart device camera.

The alignment tab edge 214 may allow user to align the alignment tab 106 of the otoscope clip device 100 such that the otoscope associated with the otoscope assembly 120 may be aligned with the smart device camera. The alignment tab edge 214 be used by the user to align the alignment tab 106 in the vertical axis and the horizontal axis of the display 103. For example, the user may place the alignment tab edge 214, on, near, or within the alignment image outline 204 and to achieve alignment. If otoscope is misaligned with the smart device camera, a portion of the alignment tab edge 214 may appear outside of the alignment image outline 204. The user may adjust the otoscope clip device 100 to address the portion of the alignment tab edge 214 that may be outside of the alignment image outline 204 and may align the otoscope with the device camera. By correcting the misalignment of the alignment tab edge 214 with the alignment image outline 204, the user may correct the alignment of the otoscope with the smart device camera.

The alignment tab edge 214 may be an edge of the alignment tab that may be parallel and may be in contact with the phone screen. The alignment tab edge 214 may fit within the alignment image 104 such that the alignment image outline 204 may be seen. The alignment tab edge 214 may be shaped to help reveal the alignment image outline 204 when the alignment tab edge 214 may be placed on top of or over the alignment image outline 204. For example, the alignment tab edge 214 may be created by tapering, chamfering, rounding, or filleting an edge of the alignment tab 106. The alignment tab edge 214 may be complementary and/or the same shape as the alignment image outline 204.

The alignment tab edge 214 may be the same color as or a similar color to alignment image 104, the alignment tab 106, and/or the alignment image outline 204. The alignment tab edge 214 may be a different color than the alignment image 104, the alignment tab 106, and/or the alignment image outline 204.

The horizontal alignment tab feature 212 may be used by a user to determine how to move the otoscope clip device 100 to achieve alignment. The horizontal alignment tab feature 212 may be a line, an object, a shape, an indicator, an icon, an etching, and/or the like. The horizontal alignment tab feature 212 may be a clear portion or a window of the alignment tab 106. The horizontal alignment tab feature 212 may be an oval elongated in the vertical axis. The horizontal alignment tab feature 212 may be a pill shape elongated along the vertical axis.

The horizontal alignment tab feature 212 may be a first arrow-shaped protrusion and a second arrow-shaped protrusion that are placed a distance from each other in a vertical direction. The first arrow-shaped protrusion and the second arrow-shaped protrusion may be designed to point at each other.

In an example, the horizontal alignment tab feature 212 may comprise a first aperture and a second aperture that are a distance from each other. The first aperture and the second aperture may be complementary to one or more objects within the alignment image 104. For example, the first aperture may be aligned with a first circle within the alignment image 104, and the second aperture may be aligned with a second circle within the alignment image 104.

The horizontal alignment tab feature 212 may be aligned with a feature that may be within the alignment image 104, may be outside the alignment image 104, and/or may intersect the alignment image outline 204. The horizontal alignment tab feature 212 may be used by a user to determine how to move the otoscope clip device 100 along a horizontal axis associated with the display 103 to horizontally align the otoscope associated with the otoscope assembly 120 with the smart device camera.

To provide guidance as to how to move the otoscope clip device 100 along the horizontal axis, the horizontal alignment tab feature 212 may have one or more reference points. For example, the horizontal alignment tab feature 212 may be a number of dots along an axis, a line along the axis, a shape elongated along the axis, an indicator along the axis, and/or the like.

To align the otoscope with the smart device camera along the horizontal axis, a user may attach the otoscope clip device 100 on the smart device 102 such that the horizontal alignment tab feature 212 may be aligned with the horizontal alignment image feature 206. For example, the user may align the arrow or triangular protrusions of the horizontal alignment tab feature 212 with the line of the horizontal alignment image feature 206.

The vertical alignment tab feature 210 may be used by a user to determine how to move the otoscope clip device 100 to achieve alignment. The vertical alignment tab feature 210 may be a line, an object, a shape, an indicator, an icon, an etching, and/or the like. The vertical alignment tab feature 210 may be a clear portion or a window of the alignment tab 106. The vertical alignment tab feature 210 may be an oval elongated along the horizontal axis. The vertical alignment tab feature 210 may be a pill shape elongated along the horizontal axis. The vertical alignment tab feature 210 may align with a feature that may be within the alignment image 104, may be outside the alignment image 104, and/or may intersect the alignment image outline 204. The vertical alignment tab feature 210 may be used by a user to determine how to move the otoscope clip device 100 along the vertical axis associated with the display 103 to vertically align the otoscope associated with the otoscope assembly 120 with the smart device camera.

In an example, the vertical alignment tab feature 210 may comprise a first arrow-shaped protrusion and a second arrow-shaped protrusion that are placed a distance from each other in a horizontal direction. The first arrow-shaped protrusion and the second arrow-shaped protrusion may be designed to point at each other.

In an example, the vertical alignment tab feature 210 may comprise a first aperture and a second aperture that are a distance from each other. The first aperture and the second aperture may be complementary to one or more objects within the alignment image 104. For example, the first aperture may be aligned with a first circle within the alignment image 104, and the second aperture may be aligned with a second circle within the alignment image 104.

To provide guidance as to how to move the otoscope clip device 100 along the vertical axis, the vertical alignment tab feature 210 may be one or more reference points. For example, the vertical alignment tab feature 210 may be a number of dots along an axis, a line along the axis, a shape elongated along the axis, an indicator along the axis, and/or the like.

To align the otoscope with the smart device camera along the vertical axis, a user may attach the otoscope clip device 100 on the smart device 102 such that the vertical alignment tab feature 210 may be aligned with vertical alignment image feature 208. For example, the user may align the oval aperture of the vertical alignment tab feature 210 with the vertical alignment image feature 208.

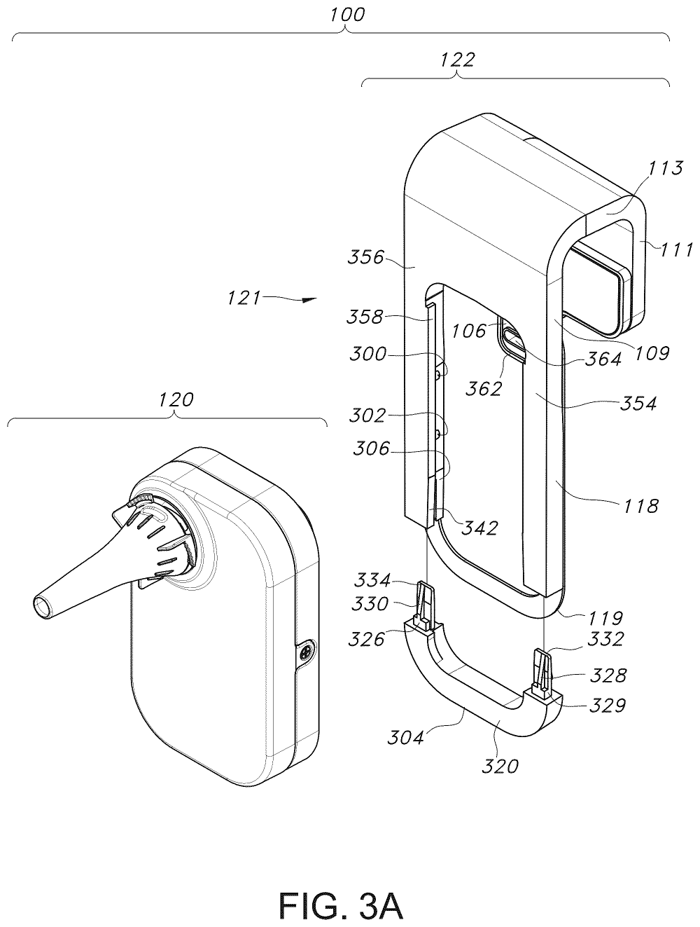

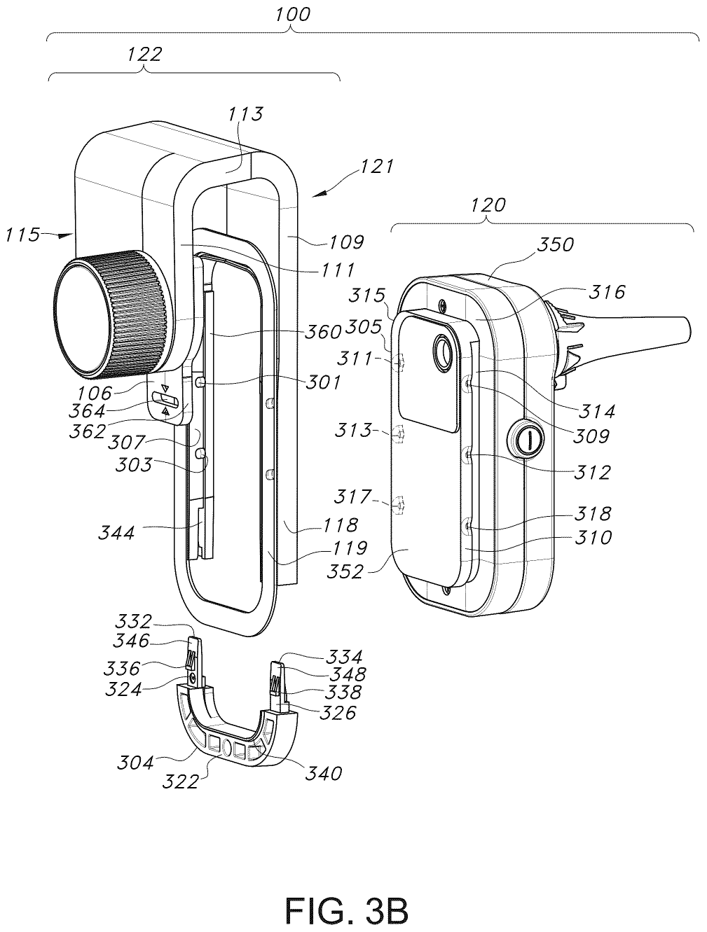

FIGS. 3A-B depict perspective views of an otoscope clip device that may comprise an otoscope assembly and a clip assembly. FIG. 3A shows a first perspective of the otoscope clip device 100, which shows the front side or device-side 121 of the otoscope clip device 100. FIG. 3B shows a second perspective of the otoscope clip device 100, which shows the back side or dial-side 115 of the otoscope clip device 100. As shown in FIGS. 3A-B, the otoscope clip device 100 may comprise the otoscope assembly 120 and the clip assembly 122.

As shown in FIG. 3B, the otoscope assembly 120 may comprise the main body 350, the otoscope back-plate 316, and the otoscope engagement member 352. The otoscope back-plate 316 may be connected to the main body 350. The otoscope engagement member 352 may be connected to the clip assembly 122. For example, the otoscope engagement member 352 may allow the otoscope assembly 120 attach to, connect to, or engage with the clip engagement member 118 of the otoscope clip device 100. The clip assembly 122 may comprise the dial-side member, 111, the top member 113, and the device-side member 109. The device-side member 109 may comprise the clip engagement member 118.

The otoscope engagement member 352 may comprise a guide track, such as guide track 314 and guide track 315. The guide track may be a channel formed in a surface of the otoscope engagement member 352 that may face an inner surface of the clip engagement member 118. The guide track may be a channel in a surface of the otoscope engagement member 352 that may be orthogonal to a surface of the otoscope back-plate 316. The guide track may be connected to the otoscope back-plate 316 and may be connected to a protruding connecting member, such as protruding connecting member 310 and/or protruding connecting member 305.

The otoscope engagement member 352 may have an undercut in one or more sides that may be orthogonal to the otoscope back-plate 316 such that an overhang may be created. The overhang may be parallel to the otoscope back-plate 316. The undercut may be the guide track 314 and/or the guide track 315. The overhang may be the protruding connecting member 310 and/or the protruding connecting member 305. The overhang may include one or more receiver elements that may be designed to engage with one or latching elements within the channel 306 and/or the channel 307. A latching element of the one or more latching elements may be a resilient member, a protrusion, an outwardly extending lug, a key, and/or the like. A receiver element of the one or more receiver elements may be a recess, a cut out, a detent indentation, and/or the like.

The protruding connecting member 310 may include the receiver element 309, which may engage with the latching element 300. The protruding connecting member 310 may include the receiver element 312, which may engage the latching element 300 and/or the latching element 302 (shown in FIG. 3A). The protruding connecting member 310 may include the receiver element 318, which may engage with the latching element 302.

The protruding connecting member 305 may include the receiver element 311, which may engage with the latching element 301. The protruding connecting member 305 may include the receiver element 313, which may engage with the latching element 301 and/or the latching element 303. The protruding connecting member 305 may include the receiver element 317, which may engage with the latching element 303.

The otoscope assembly 120 may be slidably connected to the clip assembly 122. The protruding connecting member 310 may slidable connect with the channel 306. The protruding connecting member 305 may slidable connect with the channel 307. The clip engagement member lip 360 may slidably connect with the guide track 315. The clip engagement member lip 358 may slidably connect with the guide track 314. The front surface of the engagement member leg 356 and the front surface of the engagement member leg 354 may slidably connect with the otoscope back-plate 316.

The clip assembly 122 may comprise the clip engagement member 118 and the end cap 304. The clip engagement member 118 may comprise a pair of legs extending from the ends of a connecting yolk portion that extends perpendicularly to the pair of legs. A leg (e.g., each leg) may comprise a recess at the distal end of the leg, such as the engagement member leg recess 342 and the engagement member leg recess 344, which may allow the end cap 304 to connect with and/or attach to the clip engagement member 118. A leg (e.g., each leg) may comprise a channel, such as the channel 306 and the channel 307, that cuts into a side of the leg that faces the opposing leg. The channel 307 may face the opposing leg. The channel 307 may face the channel 306. The channel 307 may form a track that may engage with the protruding connecting member 305 from the otoscope assembly 120. The channel 307 may engage with the protruding connecting member 305 from otoscope assembly 120 such that clip assembly 122 may be movably connected, attached to, or engaged with clip engagement member 118 and/or clip assembly 122. For example, the channel 307 may allow the protruding connecting member 305 to slidably move within the channel 307 while engaging with the protruding connecting member 305 such that the clip assembly 122 may remain in contact with the otoscope assembly 120.

The channel 307 may include one or more latching elements. For example, the channel 307 may include the latching element 301 and/or the latching element 303. The latching element 301 and/or the latching element 303 may be a resilient member, a protrusion, an outwardly extending lug, a key, and/or the like.

The latching element 301 and/or the latching element 303 may be protrusions or outwardly extending lugs that may be received by a recess, such as a detent indentation or a radial recess, that may be provided by the protruding connecting member 305, such as the receiver element 311, the receiver element 313, and/or the receiver element 317. A receiver element, such as the receiver element 311, the receiver element 313, and/or the receiver element 317, may be a recess, a cut out, a detent indentation, and/or the like.

In a first position, the latching element 301 may resiliently deform a surface of the protruding connecting member 305 and/or may engage with the receiver element 313, for example, in a snap fit. The latching element 303 may resiliently deform a surface of the protruding connecting member 305 and/or may engage with the receiver element 317, for example, in a snap fit.

In a second position, the latching element 301 may resiliently deform a surface of the protruding connecting member 305 and/or may engage with the receiver element 311, for example, in a snap fit. The latching element 303 may resiliently deform a surface of the protruding connecting member 305 and/or may engage with the receiver element 313, for example, in a snap fit.

The channel 306 may face the opposing leg. The channel 306 may face the channel 307. The channel 306 may form a track that may engage with the protruding connecting member 310 from the otoscope assembly 120. The channel 306 may engage with the protruding connecting member 310 from otoscope assembly 120 such that clip assembly 122 may be moveably connected, attached to, or engaged with the clip engagement member 118 and/or the clip assembly 122. For example, the channel 306 may allow the protruding connecting member 310 to slidably move within the channel 306 while engaging with the protruding connecting member 310 such that the clip assembly 122 may remain in contact with the otoscope assembly 120.

The channel 306 may include one or more latching elements. For example, the channel 306 may include the latching element 300 and/or the latching element 302. The latching element 300 and/or the latching element 302 may be a resilient member, a protrusion, an outwardly extending lug, a key, and/or the like.

The latching element 300 and/or the latching element 302 may be protrusions or outwardly extending lugs that may be received by a recess, such as a detent indentation or a radial recess, provided by protruding connecting member 310, such as the receiver element 309, the receiver element 312, and/or receiver element 318. A receiver element, such as the receiver element 309, the receiver element 312, and/or the receiver element 318, may be a recess, a cut out, a detent indentation, and/or the like.

In a first position, the latching element 300 may resiliently deform a surface of the protruding connecting member 310 and/or may engage with the receiver element 312, for example, in a snap fit. The latching element 302 may resiliently deform a surface of the protruding connecting member and/or may engage with the receiver element 318.

In a second position, the latching element 300 may resiliently deform a surface of the protruding connecting member 310 and/or may engage with the receiver element 309. The latching element 302 may resiliently deform a surface of the protruding connecting member 310 and/or may engage with receiver element 312, for example, in a snap fit.

The clip assembly 122 may comprise a contact surface 119. The contact surface 119 may be attached to a face of the clip engagement member 118, such as the face of the clip engagement member 118 that may be directed to a surface of the smart device that includes the camera. The contact surface 119 may be of an oval shape that may be complementary to the clip engagement member 118. The contact surface 119 may have an elongated aperture. The elongated aperture may match a complementary aperture in clip engagement member 118. The elongated aperture may allow a viewing portion of the otoscope assembly 120 to go through the contact surface 119. The contact surface 119 may be made of a resilient material that may absorb and/or apply pressure to a surface of the smartphone device. The contact surface 119 may be made of an anti-slip material that may prevent the otoscope clip device 100 from moving when clamped to a smartphone device. The contact surface 119 may be made of a material that avoids damage, and/or scratches to the smartphone device. For example, the contact surface may be made of rubber, plastic, a polymer, and/or the like. The contact surface 119 may act as a light block to prevent light from leaking from the otoscope clip device, may prevent light from entering the otoscope clip device optics, and/or may act as a light block to prevent light from entering a camera of the smart device.

The clip assembly 122 may comprise the end cap 304. The end cap 304 may be a u-shaped member with a pair of legs, such as the end cap leg 332 and the end cap leg 334, extending from the ends of the curved center portion. The end cap 304 may be located at the proximal end of the clip engagement member 118. The end cap 304 may comprise a front surface 320 and back surface 322. When the otoscope clip device 100 is attached to a smart device, the front surface 320 many face the otoscope assembly 120 and the back surface 322 may face a surface of the smart device.

The end cap leg 332 may contact the u-shaped portion of end cap 304. The distal end of end cap leg 332 may be beveled at beveled portion 346. The end cap leg 332 may comprise the base end 324 that may be located at the proximal end of the end cap leg 332. The base end 324 may contact the u-shape portion of end cap 304. The base end 324 may be a smaller complementary shape to a cross-section of the u-shape portion of the end cap 304, such that a shoulder may be formed at one or more surfaces that may contact the proximal end of the base end 324. The shoulder may be orthogonal to the base end 324.

The end cap leg 332 may include the spine 328. The spine 328 may be raised from a surface of the end cap leg 332. The spine 328 may contact the base end 324 and may extend towards the distal end of end cap 304. Spine 328 may have a beveled distal end. The spine 328 may have a length that may terminate at the distal end of the end cap 304 and may have a width that may be less than the width of the end cap 304. The spine 328 may be resilient and may provide a bias toward the back surface 322 to allow the end cap foot 336 to engage the engagement member leg recess 344.

The end cap leg 332 may include the end cap foot 336. The end cap foot 336 may be a projecting foot positioned near the distal end of the end cap leg 332. The end cap foot 336 may be an outwardly projecting lug and may be resilient. The end cap foot 336 may connect with a hole, recess, or cut out near the distal end of a leg of the clip engagement member 118. For example, the end cap foot 336 may connect with the engagement member leg recess 344. The engagement member leg recess 344 may be a cut out in a wall or surface of the engagement member leg recess 344 that may be orthogonal to a surface of end cap leg 332 from which end cap foot 336 may protrude from.

The end cap leg 334 may contact the u-shaped portion of end cap 304. The distal end of end cap leg 334 may be beveled at beveled portion 348. The end cap leg 334 may comprise the base end 326 that may be located at the proximal end of the end cap leg 334. The base end 326 may contact the u-shape portion of the end cap 304. The base end 326 may be a smaller complementary shape to a cross-section of the u-shape portion of the end cap 304, such that a shoulder may be formed at one or more surfaces that may connect to the proximal end of base end 326. The shoulder may be orthogonal to base end 326. The end cap leg 334 may include the spine 330.

The spine 330 may be raised from a surface of the end cap leg 334. The spine 330 may contact the base end 326 and may extend towards the distal end of the end cap 304. The spine 330 may have a beveled distal end. The spine 330 may have a length that may terminate at the distal end of the end cap 304 and may have a width that may be less than the width of end cap 304. The spine 330 may be resilient and may provide a bias toward back surface 322 to allow end cap foot 338 to engage the engagement member leg recess 344.

The end cap leg 334 may include the end cap foot 338. The end cap foot 338 may be a projecting foot positioned near the distal end of the end cap leg 334. The end cap foot 338 may be an outwardly projecting lug and may be resilient. The end cap foot 338 may connect with a hole, recess, or cut out near the distal end of a leg of the clip engagement member 118. For example, the end cap foot 338 may connect with the engagement member leg recess 342. The engagement member leg recess 342 may be a cut out in a wall or surface of the clip engagement member 118 that may be orthogonal to a surface of the end cap leg 334 from which the end cap foot 338 may protrude from.

When connected to clip engagement member 118, the end cap 304 may prevent the otoscope assembly 120 from moving beyond a position and/or disengaging with the clip assembly 122. For example, the end cap 304 may prevent a user from accidentally moving the otoscope assembly 120 beyond a point to cause damage to the otoscope assembly 120.

To assemble otoscope clip device 100, the otoscope assembly 120 may be slid into the clip assembly 122 and the end cap 304 may be connected to the clip engagement member 118 to retain otoscope assembly 120 within the clip assembly 122. The otoscope assembly 120 may slidably connect with clip assembly 122. For example, the protruding connecting member 310 may slide into the channel 306, the protruding connecting member may slide into the channel 307, the clip engagement member lip 360 may slide into the guide track 315, and the clip engagement member lip 358 may slide into the guide track 314.

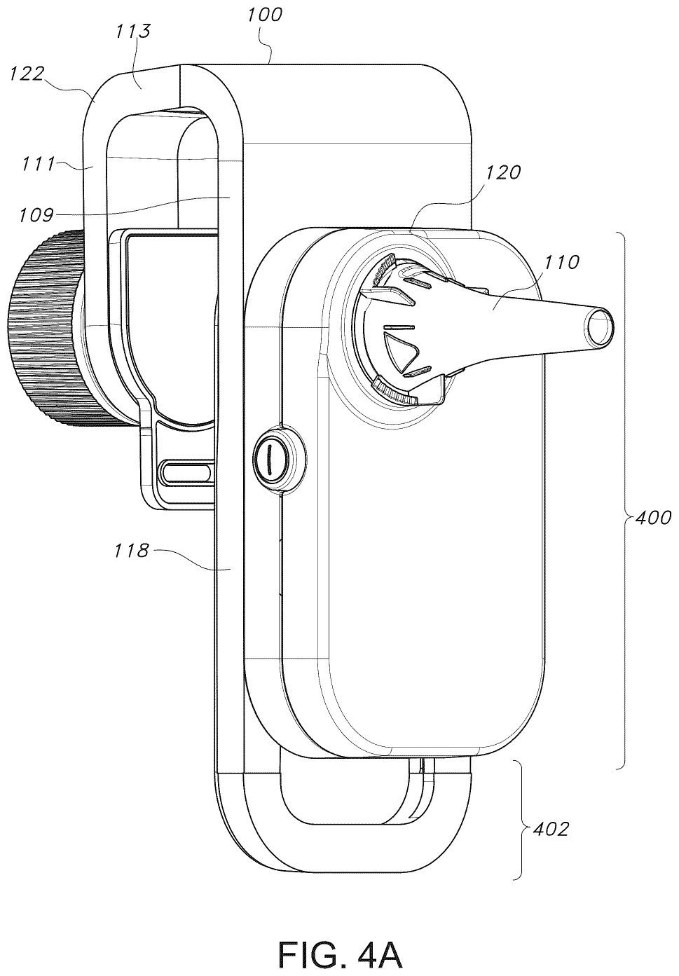

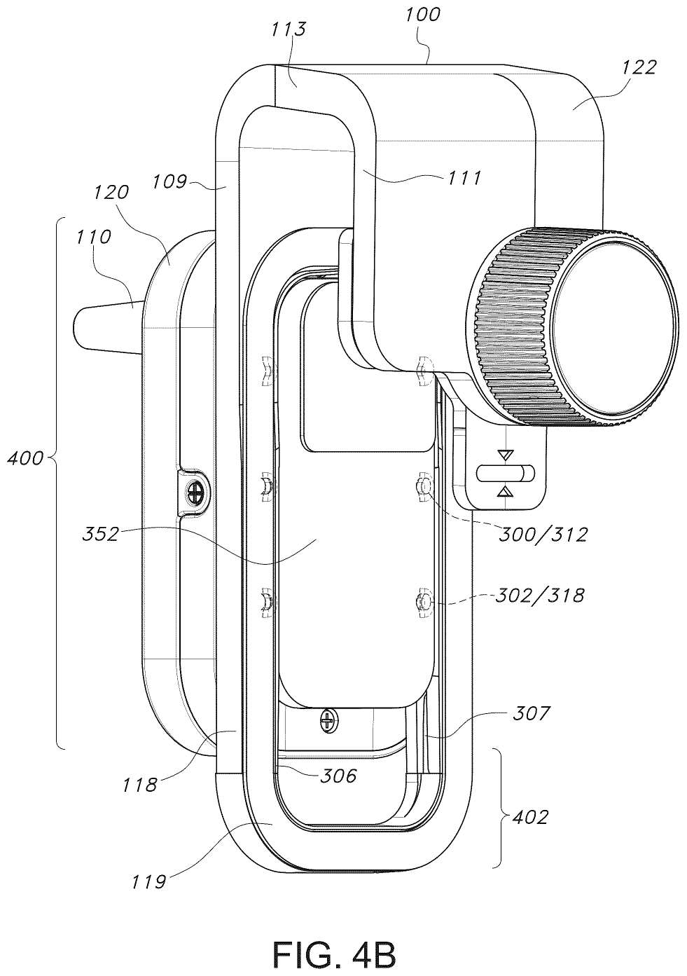

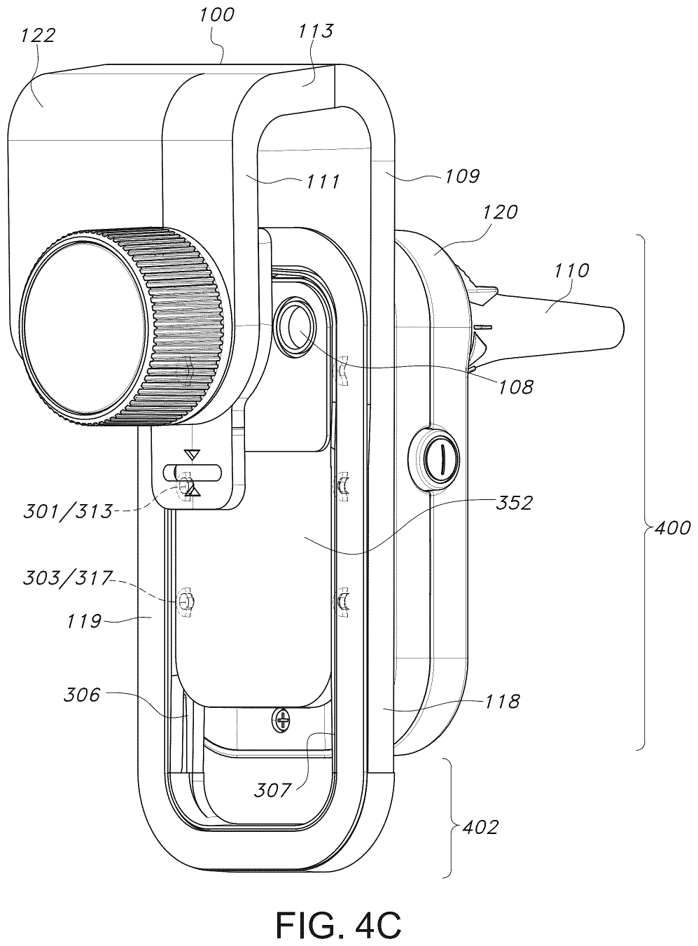

FIGS. 4A-C depict perspective views of an example otoscope clip device where the otoscope assembly is in a first position, which may be an upper position, within a clip assembly.

Different smart devices may include a camera in different positions and/or locations. For example, a first smart device may have the camera at a length from a distal end of the first smart device. A second smart device may have a camera at a length from a distal end of the second smart device that may be twice as long as the length for the first smart device.

To account for the differences in length, the otoscope clip device 100 may be adjustable. For example, the otoscope clip device 100 may allow the otoscope assembly 120 to be placed in one or more locations and/or positions within the clip assembly 122. As shown in FIGS. 4A-C, the otoscope clip device 100 may comprise the clip assembly 122 and the otoscope assembly 120. The otoscope assembly 120 may be slidably movable within the clip assembly 122. The otoscope assembly 120 may be slidably movable within the clip assembly 122 to assist in aligning the viewing portion 108 of the otoscope assembly with a smart device.

The otoscope assembly 120 may be in a first position, which may be the upper position 400, within the clip assembly 122. The upper position 400 may be an upper position within the clip assembly 122. For example, the upper position 400 may be a position where the otoscope assembly 120 may be near the proximal end of the clip engagement member 118. A user may place the otoscope assembly 120 in the upper position 400 by sliding otoscope assembly 120 toward the proximal end of the clip engagement member 118. When the otoscope assembly 120 is at the upper position 400, the otoscope engagement member 352 may create a gap in the lower portion such as the clip engagement member gap 402.

When the otoscope assembly 120 may be in the first position, the viewing portion 108, and/or the outer tip housing 110 may be aligned with the camera of the smart device. The otoscope assembly 120 may move or slide to the upper position 400 when the otoscope engagement member 352 slides toward the proximal end of the clip engagement member 118, for example, using the channel 306 and the channel 307. The otoscope assembly 120 may maintain the upper position 400 by being slidably connected to the clip engagement member 118. The otoscope assembly 120 may be slidably connected to the clip engagement member 118 at the upper position 400 via the latching element 300 being engaged to the receiver element 312, the latching element 302 being engaged to the receiver element 318, the latching element 301 being engaged to the receiver element 313, and/or the latching element 303 being engaged to the receiver element 317.

When in the upper position 400, the otoscope assembly 120 may not use one or more latching elements. The otoscope engagement member 352 may not use one or more latching elements provided by the channel 306 and/or the channel 307. For example, the receiver element 311 and/or the receiver element 309 may not be engaged with a latching element.

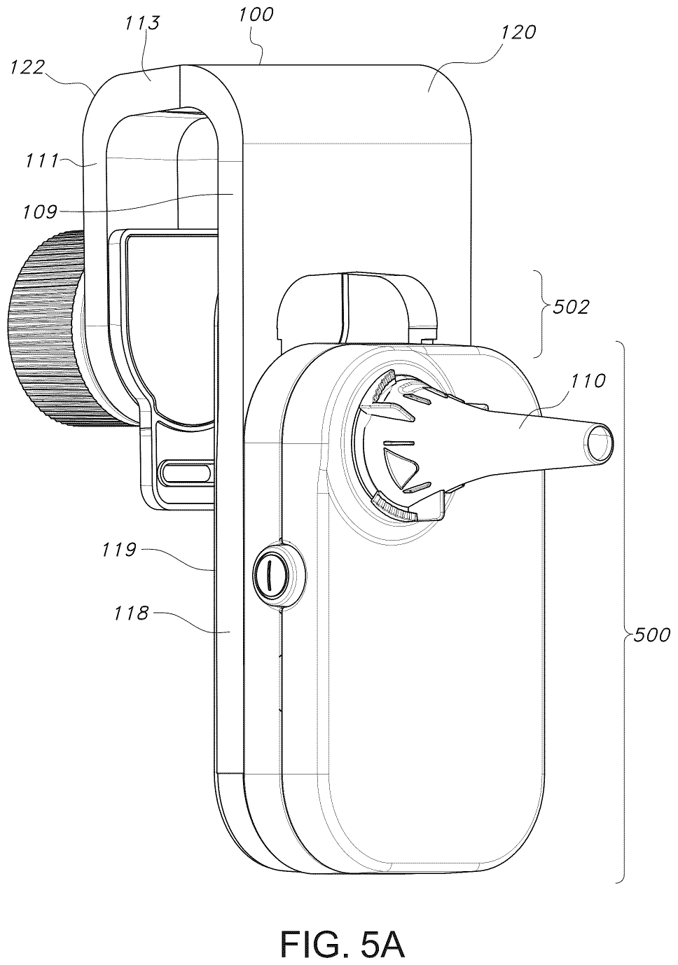

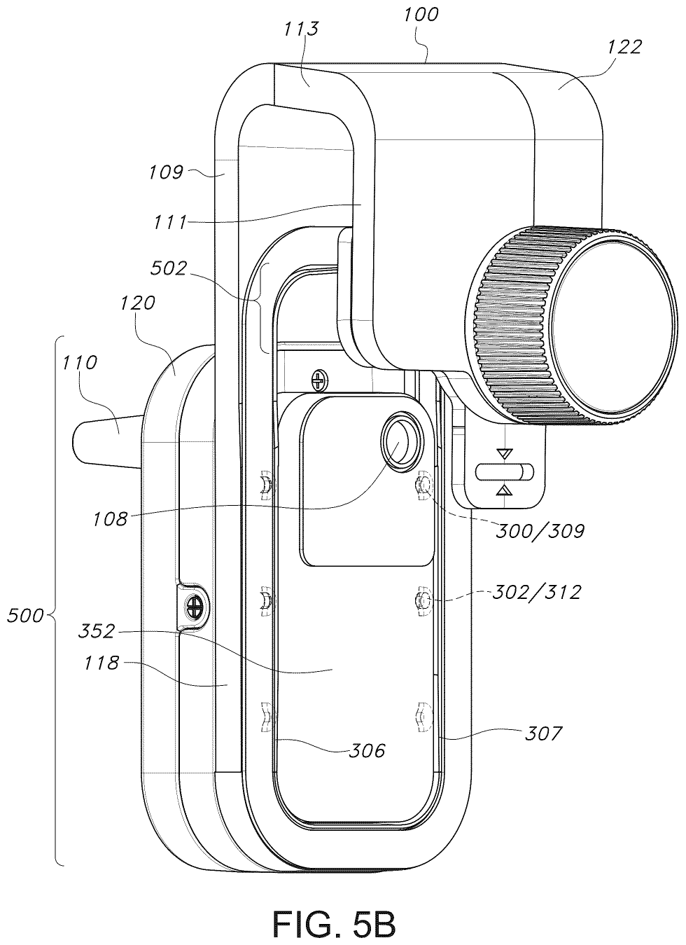

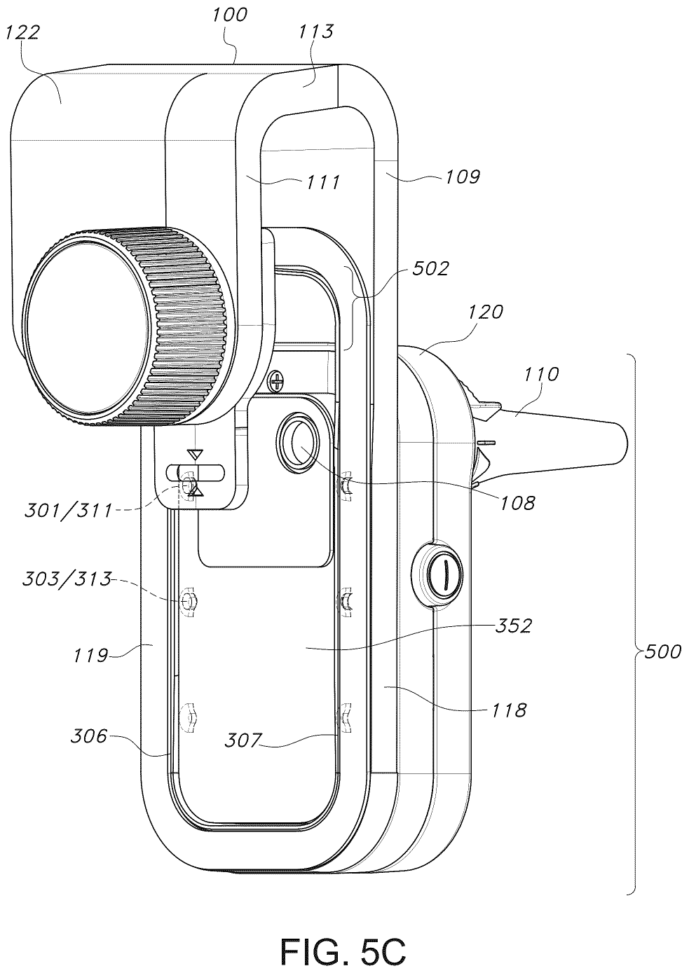

FIGS. 5A-C depict perspective views of an example otoscope clip device where the otoscope assembly is in a second position, which may be a lower position, within a clip assembly. To account for the differences in length, the otoscope clip device 100 may be adjustable. For example, the otoscope clip device 100 may allow the otoscope assembly 120 to be placed in one or more locations and/or positions within the clip assembly 122. As shown in FIGS. 5A-C, the otoscope clip device 100 may comprise the clip assembly 122 and the otoscope assembly 120. The otoscope assembly 120 may be slidably movable within clip assembly 122. The otoscope assembly 120 may be slidably movable within clip assembly 122 to assist in aligning the viewing portion 108 of the otoscope assembly with a smart device.

The otoscope assembly 120 may be in a second position, which may be the lower position 500, within the clip assembly 122. The lower position 500 may be a lower position within the clip assembly 122. For example, the lower position 500 may be a position where the otoscope assembly 120 may be near the proximal end of the clip engagement member 118. A user may place the otoscope assembly 120 in the lower position 500 by sliding otoscope assembly 120 toward the proximal end of the clip engagement member 118. When the otoscope assembly 120 is at the lower position 500, the otoscope engagement member 352 may create a gap in the upper portion such as the clip engagement member gap 502.

When the otoscope assembly 120 may be in the second position, the viewing portion 108 and/or the outer tip housing 110 may be aligned with the camera of the smart device. The otoscope assembly 120 may move or slide to the lower position 500 when otoscope engagement member 352 slides toward the distal end of the clip engagement member 118, for example, using the channel 306 and the channel 307.

The otoscope assembly 120 may maintain the lower position 500 by being slidably connected to the clip engagement member 118. The otoscope assembly 120 may be slidably connected to the clip engagement member 118 at the position 500 via the latching element 302 being engaged to the receiver element 312, the latching element 300 being engaged to the receiver element 309, the latching element 303 being engaged to the receiver element 313, and the latching element 301 being engaged to the receiver element 311.

When in the lower position 500, the otoscope assembly 120 may not use one or more available latching elements. The otoscope engagement member 352 may not use one or more latching elements provided by the channel 306 and/or the channel 307. For example, the receiver element 317 and/or the receiver element 318 may not be engaged with a latching element.

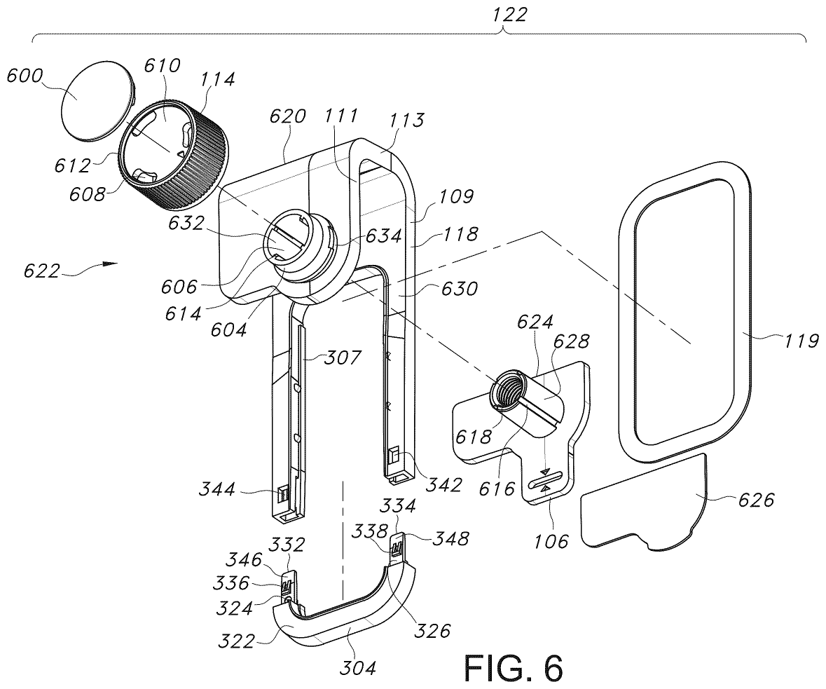

FIG. 6 depicts a schematic view of a clip assembly that may comprise one or more components. As shown in FIG. 6, the clip assembly 122 may comprise the clip engagement member 118, the connecting member 620, and the screw clamp assembly 622.

The clip engagement member 118 may comprise one or more channels that may allow for engagement and/or attachment with an otoscope engagement member. For example, the clip engagement member 118 may comprise the channel 307. The channel 307 may comprise the latching element 301 and the latching element 303. The latching element 301 and the latching element 303 may be a resilient lug or protrusion that may resiliently contact a recess that may belong to the otoscope engagement member. The clip engagement member 118 may include the engagement member leg recess 342 and the engagement member leg recess 344.

The clip engagement member 118 may comprise the end cap 304. The end cap 304 may comprise a front surface and a back surface, such as the back surface 322. The end cap 304 may comprise the end cap leg 332 and the end cap leg 334. The end cap leg 332 may comprise the beveled portion 346 that may be at the distal end of the end cap leg 332, the end cap foot 336, and the base end 324 that may be at the proximal end of the end cap leg 332. The end cap leg 334 may comprise the beveled portion 348 that may be at the distal end of the end cap leg 334, the end cap foot 338, and the base end 326 that may be at the proximal end of the end cap leg 334. The end cap 304 may be connected to the clip engagement member 118 via the end cap foot 336 engaging with engagement member leg recess 344 and end cap foot 338 engaging with the engagement member leg recess 342.

The clip engagement member 118 may comprise the inner surface 630. The inner surface 630 may be in contact with contact surface 119. The contact surface 119 may have an aperture that may be elongated to match the aperture formed within the clip engagement member 118. The contact surface 119 may be made of material that may prevent slipping, for example, when the clip assembly 122 may be attached to a smart device. The contact surface 119 may be made of a material that may prevent and/or avoid damage to a surface of a smart device. For example, the material of contact surface 119 may be in contact with the surface of a smartphone device and may prevent scratching, chipping, or other damage to surface of the smartphone device.

The connecting member 620 may be connected to the clip engagement member 118 and may be connected to the screw clamp assembly 622. The connecting member 620 may be connected to the clip engagement member 118 such that the connecting member 620 may be orthogonal to clip engagement member 118. The surfaces at which connecting member 620 may make contact the clip engagement member 118 may be rounded or may have a fillet.

The connecting member 620 may be connected to the screw clamp assembly 622 such that the connecting member 620 may be orthogonal to the screw clamp assembly 622. The surface at which connecting member 620 may contact the screw clamp assembly 622 may be rounded or may have a fillet.

The screw clamp assembly 622 may the comprise piston 624, the outer ring 604, the inner ring 606, the key 614, the aperture 632, and the knob 114. The knob 114 may be cylindrical and may have a textured outer ring which may be splines, knurling, and/or the like. The knob 114 may have a bottom side that faces the screw clamp assembly 622. The bottom side of knob 114 may have a hole that may have an outer ring, a locking ring, and a threaded protrusion in its center. The outer ring of the bottom side of the knob may have a circumference that may be larger than the outer ring 604. The locking ring may be formed at the end of the knob that is faces towards the outer ring 604. The locking ring may be of a smaller circumference than the outer ring 604. The locking ring may be of a larger circumference than locking groove 634. The locking ring may be resilient such that it may resiliently contact the locking groove 634 such that knob 114 may be movably attached to the screw clamp assembly 622.

The knob 114 may have a topside that may have one or more irregular apertures such as the aperture 608. The top side of knob 114 may comprise circular surface 610 which may be countersunk such that a lip may be formed, such as the circular lip 612. The knob cap 600 may be in contact with circular surface 610. For example, the knob cap 600 may be press fit into the circular surface 610 such that the knob cap 600 be near or flush with the circular lip 612.

The screw clamp assembly 622 may comprise the aperture 632. The aperture 632 may allow a threaded protrusion from knob 114 to contact the piston 624. The aperture 632 may be surrounded by the inner ring 606. For example, the inner ring 606 may contact a circumference of the aperture 632.

The inner ring 606 may contact a surface of the screw clamp assembly 622 that may be parallel to the clip engagement member 118. The inner ring 606 may be a hollow cylinder with the inner surface, and an outer surface, and an aperture complementary positioned to the aperture 632. The inner ring 606 may protrude a length orthogonally from a surface of the screw clamp assembly 622. The inner ring 606 may comprise one or more keys that may be located on the inner surface of the inner ring 606. For example, the inner ring 606 may comprise key 614 which may be a complementary shape to keyway 616 on shaft 628 of piston 624.

The outer ring 604 may be of a larger circumference than the inner ring 606. The outer ring 604 may be in contact with a surface of the screw clamp assembly 622. The outer ring may protrude a length orthogonally from a surface of the screw clamp assembly 622. The distal end of the outer ring 604 may be chamfered. The outer ring 604 may have one or more notches or kerfs. The kerf may begin at the distal end of the outer ring 604 and may end a length before the proximal end of the outer ring 604. The proximal end of outer ring 604 may be in contact with the locking groove 634.

The locking groove 634 may have a circumference that may be smaller than outer ring 604. The locking groove 634 may be larger than the circumference of inner ring 606. The locking groove 634 may be in contact with the outer ring 604 and a surface of screw clamp assembly 622 that may be parallel to the clip engagement member 118. The locking groove 634 may protrude a length orthogonally from a surface of the screw clamp assembly 622.

The screw clamp assembly 622 may comprise the piston 624. The piston 624 may comprise of a rectangular upper portion with rounded or filleted edge and may comprise a u-shaped bottom portion. The u-shape of the of u-shaped bottom portion may be in contact with the alignment tab 106. The piston may comprise a front surface and a parallel back surface. The back surface may be in contact with the contact surface 626. When the piston is placed in the screw clamp assembly 622, the front surface and the back surface of the piston may be parallel to a surface of the screw clamp assembly 622 and the clip engagement member 118. The front surface may be connected to the shaft 628.

The shaft 628 may protrude orthogonally from the front surface of the piston 624. The distal end of the shaft 628 may have a threaded hole, such as threaded hole 618. The shaft 628 may have one or more keyways, such as keyway 616. The keyway 616 may have a complementary shape to the key 614. The keyway 616 may be a channel that runs from the distal end of the shaft 628 to the proximal end of the shaft 628 and may terminate at the front surface of the piston 624.

When assembled, the screw clamp assembly 622 may engage the piston 624 such that the contact surface 626 and the contact surface 119 may become pads of a clamp. The screw clamp assembly 622 may cause the piston 624 to move towards the clip engagement member 118, such that the contact surface 626 may contact one surface of a smart device and the contact surface 119 may contact another surface of the smart device.

The contact surface 626 and/or the contact surface 119 made up a material that prevents damage, such as scratching, to a surface of the smart device. The contact surface 626 and/or the contact surface 119 may be made of rubber, fabric, plastic, and/or any other material that may prevents damage to the smartphone. The contact surface 626 and/or the contact surface 119 may be made of a material that prevents the clamp from slipping from the surface of the smartphone. For example, the contact surface 626 and/or the contact surface 119 may be made of rubber which may provide friction to avoid the clamp from slipping and/or moving from a position on a surface of the smart device.

The screw clamp assembly 622 may be assembled. The end cap 304 may be in contact with the clip engagement member 118. The contact surface 119 may be placed on the inner surface 630 of clip engagement member 118. The inner surface 630 may face a surface of the smart device that includes a camera. The inner surface 630 may be parallel to the inner surface of the screw clamp assembly.

The contact surface 626 may be in contact with the piston 624. The shaft 628 of the piston 624 may be placed within the aperture formed by inner ring 606 such that keyway 616 may contact the key 614. For example, the shaft 628 of the piston 624 may be placed within aperture 632. When the shaft 628 is placed through the aperture 632, the piston 624 may be positioned such that contact surface 626 may be parallel to the contact surface 119 and the alignment tab 106 may extend beyond an edge of the screw clamp assembly 622.

The knob cap 600 may be placed in contact with the circular surface 610, which may be associated with knob 114. For example, the knob cap 600 may be press fit into circular surface 610 such that the knob cap 600 may be in contact with (e.g., flush) with circular lip 612. The knob 114 may be in contact with the outer ring 604. For example, the knob 114 may be pressed on to the outer ring 604 and may contact the interior of the knob 114 such that knob 114 may be moveably connected to the outer ring 604. For example, when movably connected to the outer ring 604, the knob 114 may be twisted by a user.

The knob 114 may include a threaded protrusion that may be connected to the shaft 628 of the piston 624. The threaded protrusion on the knob 114 may be inserted and/or threaded to the threaded hole 618 within the shaft 628. The threaded protrusion may allow knob 114 to secure the piston 624 in a position that may be parallel to the clip engagement member 118.

The knob 114 may be twisted and may cause the clamp formed by the screw clamp assembly 622 to clamp a smart device. For example, twisting the knob 114 may cause the threaded protrusion of the knob 114 to thread or unthread the threaded hole 618. The rotational force of the threading or unthreading the threaded hole 618 may be redirected by one or more keys onto one or more keyways such that the piston 624 may be prevented from rotating and may be forced to move linearly in a direction toward or away from the clip engagement member 118. For example, the rotational force generated by the threading or unthreading the threaded hole 618 may be transferred as a linear force to the piston 624 via the threaded hole 618, the key 614, and/or the keyway 616. Twisting the knob 114 may move the piston 624 toward clip engagement member 118, such that the contact surface 626 may move towards a parallel surface of the clip engagement member 118, such as the contact surface 119. Twisting the knob 114 may cause the contact surface 626 and/or the contact surface 119 to contact a surface of a smartphone device. For example, twisting the knob 114 may cause the contact surface 119 to contact a back side of a smart device that may include a camera, and twisting the knob 114 may cause the contact surface 626 to contact a front side of a smart device that may include a display. The knob 114 may comprise a thread, such as a reverse thread.

When turned, the knob 114 may engage the screw clamp assembly 622 such that the piston 624 that may include alignment tab 106 may be moved towards a surface that may be parallel to alignment tab 106, such as the contact surface 119 that may be in contact with a surface of the clip engagement member 118. For example, a user may turn the knob 114 in a clockwise direction so that the alignment tab 106 may move towards a parallel surface of the clip assembly 122. The knob 114 may cause the alignment tab 106 to move towards the clip engagement member 118 such that alignment tab 106 and the clip engagement member 118 may clamp onto the smart device. A user may turn the knob 114 in a counterclockwise direction so that the alignment tab 106 may move away from a parallel surface of the clip assembly 122.

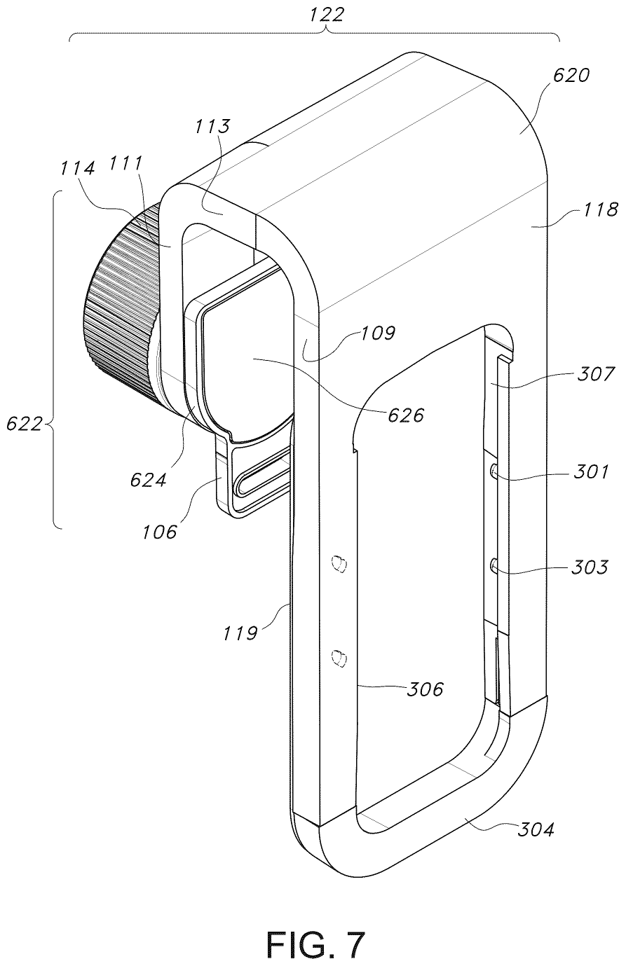

FIG. 7 depicts a perspective view of a clip assembly that may comprise a screw clamp assembly. As shown in FIG. 7, the clip assembly 122 may be assembled and may comprise a number of components. The clip assembly 122 may comprise the knob 114. The knob 114 may be connected to the piston 624 via an aperture through the screw clamp assembly 622, such as aperture 632 (shown in FIG. 6). The piston 624 may be in contact with the contact surface 626. The piston 624 may comprise the alignment tab 106. The screw clamp assembly 622 may be in contact with the connecting member 620. The connecting member 620 may be in contact with the clip engagement member 118. The clip engagement member 118 may comprise an elongated aperture and may have one or more internal sidewalls along the elongated aperture. The one or more internal sidewalls of the clip engagement member 118 may comprise a channel. For example, the clip engagement member 118 may comprise the channel 306 and the channel 307. The channel 307 may comprise the latching element 301 and the latching element 303. The channel 306 may comprise the latching element 300 and the latching element 302. The clip engagement member 118 may comprise the end cap 304.

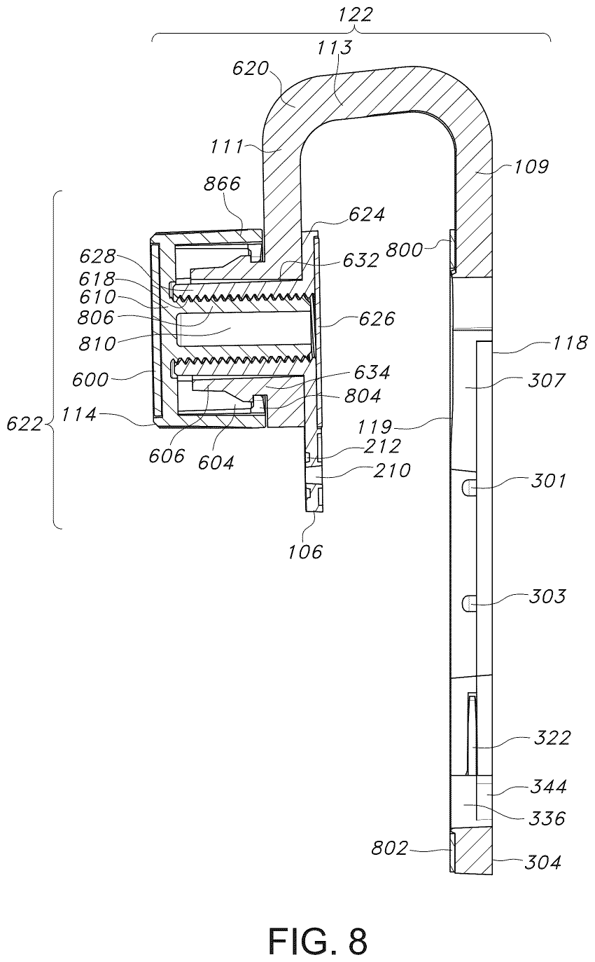

FIG. 8 depicts a cross section view of a clip assembly that may comprise a screw clamp assembly. The clip assembly 122 may comprise the clip engagement member 118. The clip engagement member 118 may comprise the channel 307 that may run a length of the clip engagement member 118. The channel 307 may comprise the latching element 301 and the latching element 303. The latching element 301 and/or the latching element 303 may engage a receiver element that may belong to an otoscope engagement member.

The clip engagement member 118 may comprise the engagement member leg recess 344. The engagement member leg recess 344 may be engaged with the end cap foot 366. The end cap foot 366 may be resilient and may be resiliently connected to the engagement member leg recess 344, such that end cap 304 may be removably attached to clip engagement member 118.

The clip engagement member 118 may be connected to the contact surface 119. The contact surface 119 may be of varying thickness. For example, the contact surface 119 may be of one thickness at its center and may be at a different thickness at its ends. For example, the contact surface 119 may have a different thickness at the end of contact surface 800 and/or the end of contact surface 802 than at a different location within the contact surface 119.

The screw clamp assembly 622 may comprise the aperture 632. The aperture 632 located at a surface of the screw clamp assembly 622 that may be parallel to the clip engagement member 118. The shaft 628 of the piston 624 may pass through the aperture 632. The shaft 628 may be connected to the piston 624. The shaft of 628 may protrude orthogonally from a surface of the piston 624. The shaft of 628 may move freely through the aperture 632. The shaft of 628 may have an exterior and an interior. The interior of the shaft of 628 may comprise the threaded hole 618. For example, the distal end of the shaft 628 may have a threaded hole.

The threaded hole 618 may be engaged by the knob 114 via the threaded protrusion 806. For example, the threaded protrusion 806 may contact the threaded hole 618 by threading the threaded hole 618. The threaded protrusion 806 may have a hollow interior. For example, the threaded protrusion 806 may have an outer wall with a thread and may have an inner wall that may form a cylindrical hole that extends towards the piston 624, such as the hole 810. The threaded protrusion 806 may have a reverse thread. The threaded protrusion 806 may protrude from the circular surface 610. For example, the threaded protrusion 806 may protrude orthogonally from the circular surface 610. The threaded protrusion 806 may be connected to the circular surface 610.

The circular surface 610 may be countersunk such that a circular lip may be formed. The knob cap 600 may be in contact with circular surface 610. For example, the knob cap 600 may be press fit into circular surface 610 such that knob cap 600 be near or flush with the circular lip.

The aperture 632 may be surrounded by the inner ring 606. For example, the inner ring 606 may contact a circumference of the aperture 632.

The inner ring 606 may contact a surface of the screw clamp assembly 622 that may be parallel to the clip engagement member 118. The inner ring 606 may be a hollow cylinder with the inner surface, and an outer surface, and an aperture complementary positioned to the aperture 632. The inner ring 606 may protrude a length orthogonally from a surface of the screw clamp assembly 622. The inner ring 606 may comprise one or more keys that may be located on the inner surface of the inner ring 606.

The outer ring 604 may be of a larger circumference than the inner ring 606. The outer ring 604 may be in contact with a surface of the screw clamp assembly 622. The outer ring may protrude a length orthogonally from a surface of the screw clamp assembly 622. The distal end of outer ring 604 may be chamfered. The outer ring 604 may have one or more notches or kerfs. The proximal end of outer ring 604 may be in contact with the locking groove 634.

The locking groove 634 may have a circumference that may be smaller than the outer ring 604. The locking groove 643 may be larger than the circumference of the inner ring 606. The locking groove 634 may be in contact with the outer ring 604 and a surface of the screw clamp assembly 622 that may be parallel to the clip engagement member 118. The locking groove 634 may protrude a length orthogonally from a surface of the screw clamp assembly 622.

The knob 114 may comprise the circular surface 610, the threaded protrusion 806, the hole 810, the knob cap 600, and the locking ring 804. The knob 114 may be cylindrical and may have a textured outer surface, such as the knob outer surface 807, which may be splines, knurling, and/or the like. The knob 114 comprise the knob outer surface 807, the locking ring 804, and the threaded protrusion 806. The locking ring 804 may have a circumference that may be larger than the outer ring 604. The locking ring 804 may be formed at the end of the knob that is nearest to outer ring 604. The locking ring may be of a smaller circumference than the outer ring 604. The locking ring may be of a larger circumference than locking groove 634. The locking ring may be resilient such that it may resiliently contact the locking groove 634 such that knob 114 may be removably attached to the screw clamp assembly 622.

The piston 624 may comprise the alignment tab 106. The alignment tab 106 may comprise the vertical alignment tab feature 210 and the horizontal alignment tab feature 212. The vertical alignment tab feature 210 may be aperture, such as an oval aperture. The horizontal alignment tab feature 212 may be a protrusion, such as a triangle or arrow shaped protrusion. The horizontal alignment tab feature 212 may protrude orthogonally from a surface of the piston 624.

The knob 114 may be twisted and may cause the clamp formed by the screw clamp assembly 622 to clamp a smart device. For example, twisting the knob 114 may cause the threaded protrusion 806 to thread or unthread the threaded hole 618. The rotational force of the threading or unthreading the threaded hole 618 may be redirected by one or more keys onto one or more keyways such that piston 624 may be prevented from rotating and may be forced to move linearly in a direction toward or away from the clip engagement member 118. For example, the rotational force generated by the threading or unthreading the threaded hole 618 may be transferred as a linear force to piston 624 via threaded hole 618, a key, and/or a keyway. Twisting the knob 114 may move piston 624 toward the clip engagement member 118 such that the contact surface 626 may move towards the contact surface 119. Twisting the knob 114 may cause the contact surface 626 and/or the contact surface 119 to contact a surface of a smartphone device. For example, twisting the knob 114 may cause the contact surface 119 to contact a back side of a smart device that may include a camera, and twisting the knob 114 may cause the contact surface 626 to contact a front side of a smart device that may include a display.

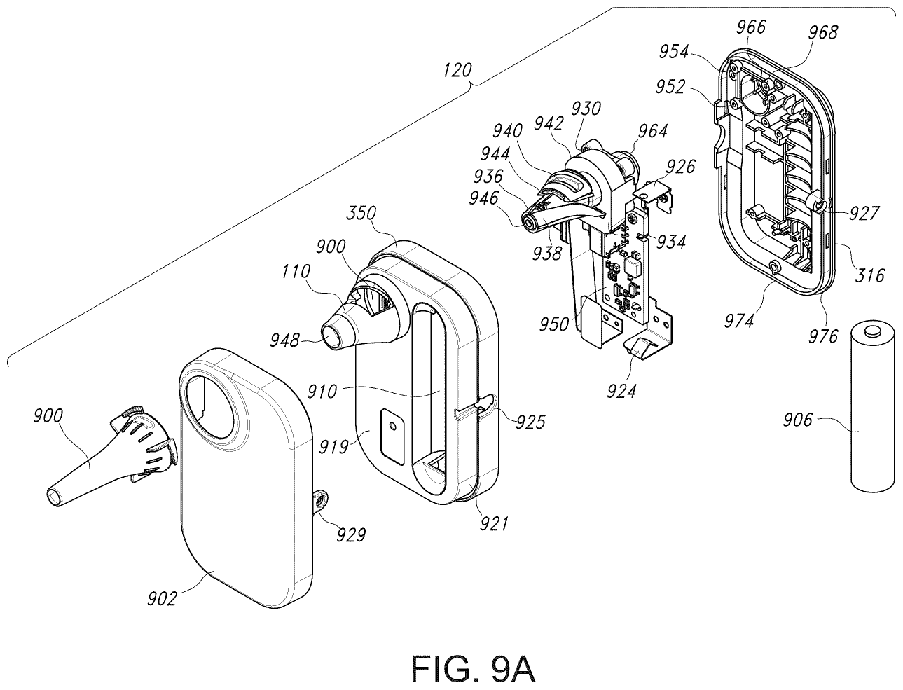

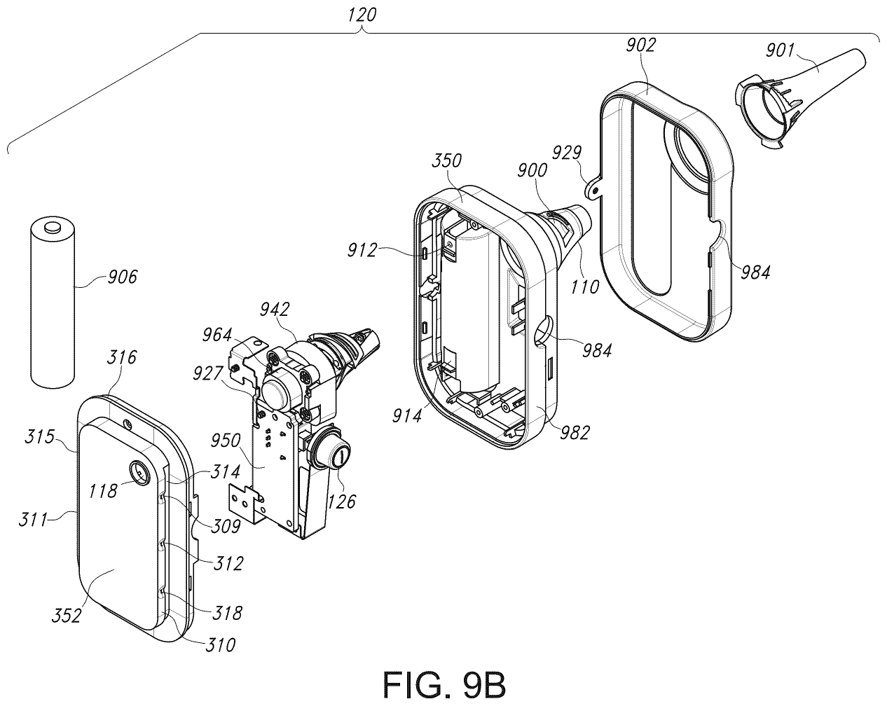

FIGS. 9A-B depict schematic views of an otoscope assembly that comprises one or more components. The otoscope assembly 120 may comprise the cover 902, the main body 350, the inner otoscope housing 942, and the otoscope back-plate 316.

The cover 902 may cover a portion of the main body 350. The cover 902 may include an aperture that may allow speculum 901 to be attached to the outer tip housing 110 through the cover 902. The cover 902 may cover the battery cavity 910 in the main body 350 that may contain the battery 906. The battery 906 and/or the battery 908 may be a battery such as a lithium ion battery, an alkaline battery, and/or the like. The cover 902 may have a top side and an underside. The underside of the cover 902 may face the battery cavity 910. Although the embodiments disclosed herein may be shown using a AAA battery, other battery shapes and/or sizes may be used. For example, the main body 350 may be reconfigured to be used with an A battery, an AA battery, an AAA battery, and/or the like.

The cover 902 may comprise the screw guide 929. The screw guide 929 may be connected to the cover 902. The screw guide 929 may include a clip that may retain a screw. For example, the screw guide 929 may retain a screw when the screw is not engaged with the screw boss 927. The cover 902 may comprise the switch opening 984, which may be a rectangular shape and may complement the light switch 126.

The screw guide 929 may protrude orthogonally from the cover 902. The distal end of the screw guide 929 may be rounded or may be a half circle. The proximal end of the screw guide 929 may be a rectangular shape and may have a surface in contact with the cover 902. The screw guide 929 may have an aperture. The aperture of the screw guide 929 may be along an axis that may be parallel to the underside surface of the cover 902. The aperture of the screw guide 929 may allow a screw to go through the screw guide 929 and contact the screw boss 927. The cover 902 may be connected to the main body 350 and may be screwed into place via a screw that goes through an aperture of the screw guide 929 and connects with the screw boss 927.

The main body 350 may comprise of an upper portion and a lower portion. The upper portion of the main body 350 may comprise the outer tip housing 110. The outer tip housing 110 may be a conical shape and may comprise an opening at the distal end such as the outer tip hole 948. The sidewall of the outer tip housing 110 may comprise a radial slot out such as the radial slot 900. The radial slot 900 may be a radial aperture such that radial protrusion 940 may go through the radial slot 900. The radial slot 900 and/or the radial protrusion 940 may allow for a speculum, such as speculum 901, to be removably connected to the outer tip housing 110.

The outer tip housing 110 may be conically hollow inside. For example, the outer tip housing 110 may be conically hollow such that the inner tip housing 944 may fit inside the outer tip housing 110. The outer tip housing 110 may have a complementary shape to the inner tip housing 944.

The main body 350 may comprise a lower portion. The lower portion of the main body 350 may comprise the battery cavity 910. The battery cavity 910 may comprise the aperture 912 and the aperture 914. The aperture 912 may allow a portion of the biased member 926 to pass through such that the biased member 926 may contact the battery 906 when the battery 906 is within the battery cavity 910. The aperture 914 may allow a portion of the biased member 924 to pass through such that the biased member 924 may contact the battery 908 when the battery 908 is within the battery cavity 910.

The main body 350 may comprise an outer sidewall, such as the main body outer sidewall 982. The main body outer sidewall 982 may comprise an opening, such as the switch opening 984 and the screw guide opening 925.

The inner otoscope housing 942 may fit inside the main body 350. The inner otoscope housing 942 may comprise the inner tip housing 944, the otoscope tube 964, and the backer board 950. The inner otoscope housing 942 may be connected to the otoscope back-plate 316 using a number of screws. For example, screw 930 may engage screw boss 954.

The inner otoscope housing 942 may comprise a number of electronics. For example, the inner otoscope housing 942 may include a printed circuit board (PCB); a light source, such as an LED; a speaker; a lens; a combination thereof; and/or the like. The otoscope tube 964 may contact and/or be attached to the inner otoscope housing 942. The proximal end of the otoscope tube 964 may contact the inner otoscope housing 942.

The otoscope tube 964 may protrude from the inner otoscope housing 942 towards the otoscope back-plate 316. The otoscope tube 964 may comprise a cylindrical protrusion with an outer wall, a hole at a distal end, and an inner wall formed by the hole. The outer wall of the otoscope tube 964 may have a smaller diameter than the aperture 968 in the otoscope back-plate 316. The otoscope tube 964 may pass through or protrude into the aperture 968 such that the otoscope tube 964 may deliver an image from the inner tip hole 946 to the viewing portion 108 via the aperture 968.

The inner otoscope housing 942 may comprise the inner tip housing 944. The inner tip housing 944 may be connected to the light pipe 938. The light pipe 938 may provide light, for example, by transporting light from an LED within the inner otoscope housing 942 towards the inner tip hole 946. The light pipe 938 may be made the plastic, fiber optic, and/or another light carrying material.

The inner otoscope housing 942 may comprise the radial protrusion 940. The radial protrusion 940 may allow a removable speculum to be removably connected to the outer tip housing 110. The radial protrusion 940 may protrude orthogonally from an outer surface of the inner tip housing 944. The radial protrusion 940 may follow a radius along the outer surface of the inner tip housing 944.

The backer board 950 may connect to a bottom portion of the inner otoscope housing 942. The backer board 950 may comprise the biased member 924 and the biased member 926. The biased member 924 and/or the biased member 926 may be made of a resilient material and may be made of an electrically conducting material. For example, the biased member 924 and/or the biased member 926 may be made of copper.

A portion of the biased member 926 may pass through the aperture 912 such that the biased member 926 may contact the battery 906 when the battery 906 is within the battery cavity 910. A portion of the biased member 924 may to pass through the aperture 914 such that the biased member 924 may contact the battery 908 when the battery 908 is within the battery cavity 910.

The otoscope back-plate 316 may be connected to the main body 350. The otoscope back-plate 316 may be removably connected to the main body 350. For example, otoscope back-plate 316 may be resiliently connected to the main body 350 using hooks and tabs such that the otoscope back-plate 316 may be removably connected to the main body 350.

The otoscope back-plate 316 may comprise the screw boss 927. The screw boss 927 may receive a screw that may be placed through the screw guide 929 and screw guide opening 925. The cover 902 may be connected to the main body 350 using a screw that may be placed through the screw guide 929 such that the screw connects with the screw boss 927.

The otoscope back-plate 316 may comprise back-plate shoulder 976. The back-plate shoulder 976 may be parallel to the otoscope engagement member 352. The back-plate shoulder 976 may connect with the back-plate lip 974. The back-plate lip 974 may protrude orthogonally from the back-plate shoulder 976. The back-plate lip 974 may fit within the main body 350 when the otoscope back-plate 316 is connected to the main body 350. The back-plate shoulder 976 may contact the main body 350 when the otoscope back-plate 316 is connected to the main body 350.

The otoscope engagement member 352 may be connected to the otoscope engagement member 352. The otoscope engagement member 352 may allow the otoscope assembly 120 attach to, connect to, or engage with a clip engagement member of an otoscope clip.

The otoscope engagement member 352 may comprise a guide track, such as the guide track 314 and the guide track 315. The guide track may be a channel formed in a surface of the otoscope engagement member 352 that may face an inner surface of a clip engagement member. The guide track may be a channel in a surface of the otoscope engagement member 352 that may be orthogonal to a surface of the otoscope back-plate 316. The guide track may be connected to the otoscope back-plate 316 and may be connected a protruding connecting member, such as the protruding connecting member 310 and/or the protruding connecting member 305.

The otoscope engagement member 352 may have an undercut in one or more sides that may be orthogonal to the otoscope back-plate 316 such that an overhang is created. The overhang may be parallel to the otoscope back-plate 316. The undercut may be the guide track 314 and/or the guide track 315. The overhang may be the protruding connecting member 310 and/or the protruding connecting member 305. The overhang may include one or more receiver elements that may be designed to engage with one or more resilient members, such as latching elements within the channel 306 and/or the channel 307. The one or more receiver elements may be recesses, cut outs, detent indentation, and/or the like. For example, the protruding connecting member 310 may include receiver elements, such as the receiver element 309, the receiver element 312, and the receiver element 318. The receiver elements may engage with the latching element 300 and/or the latching element 302. As another example, the protruding connecting member 305 may include receiver elements, such as the receiver element 311, the receiver element 313, and the receiver element 317. The receiver elements may engage with the latching element 301 and/or the latching element 303.

FIG. 10 depicts a side profile view of an angled clip assembly that comprises one or more components. The angled clip assembly 1000 may be or may comprise the clip assembly 122. The angled clip assembly 1000 may comprise the device-side member 109, the dial-side member 111, the top member 113, the contact surface 119, the dial-side angle 1010, and the device-side angle 1020.

Alignment may allow an otoscope to function correctly when used with a smartphone's camera. Misalignment between the otoscope and the camera may result in suboptimal optical synchronization, impairing the otoscope's functionality or, at the very least, compromising the quality of images it can produce. Further, the diverse geometries of smartphone cameras pose challenges that may lead to misalignment.

For example, the presence of camera bumps, where the camera protrudes from the smart device's back surface, on smart devices has become prevalent. The presence of a camera bump may lead to a deformation of a clip assembly when pressure is applied to a localized area of the clip during attachment. This deformation may result in misalignment of the otoscope and the smart device camera as the deformed clip can no longer hold the otoscope in the correct position relative to the camera, leading to suboptimal image quality or the inability to use the otoscope effectively.

The deformation of the clip, resulting from uneven pressure during attachment, may lead to issues impacting the alignment between the otoscope and the smart device camera: tilt deformation and deflection deformation.

Tilt deformation may be the angular misalignment that occurs when a part of the clip, such as the device-side member 109 and/or the contact surface 119, becomes angled away from its intended orientation with respect to the camera surface. In an example, the intended origination may be parallel or approximately parallel with respect to the camera surface. This deviation may happen when the surface of the camera bump places uneven pressure on the clip assembly, leading to improper positioning of the otoscope. In an example, a camera surface may cause a force to be focused on the contact point 1030, which may cause the device-side member 109 and/or the contact surface 119 to become tilted or angled away from an intended orientation.

Deflection deformation may refer to the bending or warping of the clip assembly in areas that may be subjected to concentrated pressure. A distance of deflection, or deflection distance, may indicate how a member of the clip may move from an intended position under stress. For example, pressure at the contact point 1030 may cause deflection deformation to the device-side member 109 such that the distal end of the device-side member 109 may a deflected 7 mm (e.g., a deflection distance) from the back-side of the smart device. In the context of the clip assembly, deflection deformation may move an otoscope away from its desired position relative to the camera's optical axis.

Tilt deformation and deflection deformation may be interrelated. For example, tilt deformation may be a result of a physical change inflicted by deflection. When the clip deforms, it may not be able to maintain the otoscope in a stable, aligned position, causing the otoscope to tilt away from an optimal alignment angle. This misalignment may reduce the effectiveness of the otoscope, leading to suboptimal image quality or even rendering the device unusable. For example, the device-side member 109 of the angle clip assembly 1000 may begin to tilt away from a camera surface as pressure is applied to the contact point 1030, which may cause the distal end of the device-side member 109 to deflect away from the smart device. As another example, the pressure applied at the contact point 1030 may also result in the contact surface 119 deviating from its preferable state of being flush or nearly flush with the camera surface for optimal alignment, causing it to angle or tilt, starting at the contact point 1030 and leading to the end deflection 1040.

The angled clip assembly 1000 may minimize deflection, such as deflection caused by camera bumps. For example, the geometry of the angled clip assembly 1000 may compensate for the uneven surface created by the camera bump by allowing for an adaptable and flexible connection between the otoscope and the smart device. This adaptability may ensure that pressure is distributed more evenly across the clip, reducing the likelihood of deformation and maintaining alignment between the otoscope and the camera within a tolerance. Even with the presence of a camera bump, the angled clip assembly 1000 may ensure that the otoscope stays aligned within tolerance to minimize deflection, tilt, and/or deformation while preserving the image quality.

In an example, the angled clip assembly 1000 may be able to minimize the end deflection 1040 to a distance of between 1 mm to 3.0 mm. Minimizing deflection to a range of between 1 mm to 3 mm may enhance the alignment between interconnected devices by allowing the assembly to adapt to minor surface irregularities and variations in attachment force without causing substantial misalignment. Such controlled deflection ensures that devices, such as aligning an otoscope with a smartphone's camera, remain positioned for functionality and performance, even under external pressures or when faced with deviations in device geometry.

The geometry of angled clip assembly 1000 may contribute to minimizing tilt deformation and/or deflection deformation. The geometry may refer to the design, configuration, or arrangement of members (e.g., components) within the angled clip assembly 1000. The members may include the device-side member 109, the dial-side member 111, the top member 113, and the angles at which these components are oriented, such as the dial-side angle 1010 and the device-side angle 1020. The configuration of these components (e.g., members) and their respective angles may impact how the assembly engages with and attaches to the surface of a smart device, ensuring a secure and aligned connection. For example, the configuration of these components or members may affect the flex experienced at the device-side angle 1020 and/or the dial-side angle 1010 to minimize tilt deformation and/or deflection deformation.

The material chosen for the angled clip assembly 1000, or any component thereof, may be selected to reduce tilt and/or deflection deformations. For example, materials known for their stiffness and/or yield strength may be utilized to enhance resistance against both tilt and deflection. A higher infill density in these materials may contribute to increased stiffness, and may also reduce the likelihood of deformation. Suitable materials include advanced composites, plastics, reinforced plastics, or a combination thereof, and the like. These materials may be chosen for their ability to provide performance improvements without considerably increasing the weight or thickness of the angled clip assembly 1000. Examples of such materials include Polycarbonate-Acrylonitrile Butadiene Styrene (PC ABS) or glass-filled PC ABS (PC ABS G). For example, the material of the angled clip assembly 1000, or any component thereof, may affect the flex experienced at the device-side angle 1020 and/or the dial-side angle 1010 to minimize tilt deformation and/or deflection deformation.

Table 1 shows a comparison of how the deflection behavior may vary across different materials with varying strength (e.g., flexural modulus), illustrating how material choice may impact deflection even when adjustments are made to the geometry, such as the inset angle, of the assembly.

TABLE 1

| | | | | Deflection | Tilt or |
|---|---|---|---|---|---|
| | | | | with 1 | Deflection |
| | Flexural | | Deflection | deg inset | Angle |
| Material | Modulus | | (mm) | (mm) | (degs.) |
| PC ABS | 2.6 | GPa | 6.8 | 5.1 | 3.1 |
| PC ABS 10% | 4.25 | GPa | 4.2 | 2.5 | 1.5 |
| Glass Filled | | | | | |
| PC ABS 20% | 5.4 | GPa | 3.4 | 1.7 | 1 |
| Glass Filled | | | | | |

Structural reinforcements may be integrated into the angled clip assembly 1000, or any component thereof, to minimize tilt and/or deflection deformations. For example, although not shown in FIG. 10, the top member 113 may include ribs or gussets to enhance its rigidity and resistance to bending forces. These reinforcements may provide support to areas that are vulnerable to deflection or tilt, thereby improving the overall structural integrity and performance of the angled clip assembly. This incorporation of reinforcements may be applied to other components such as the device-side member 109 and the dial-side member 111. For example, the top member 113 may include a rib to reduce the flex experienced at the device-side angle 1020 and/or the dial-side angle 1010 when the angled clip assembly 1000 is under a load, which may minimize tilt deformation and/or deflection deformation.

Adjustments in geometry may be integrated into the angled clip assembly 1000, or any component thereof, to minimize tilt and/or deflection deformations. For example, although not shown in FIG. 10, the geometry of top member 113 may include shapes, such as curves, which have a resistance to deformation. These geometric configurations (e.g., shapes) may aid in distributing stress more evenly across the member, diminishing the likelihood of localized bending that may contribute to tilt or deflection deformation. For example, the device-side member 109 may be designed to curve towards the back-side of a smart device when the angled clip assembly 1000 is attached to the smart device, which may minimize tilt deformation and/or deflection deformation.

Stress relief features may be integrated into the angled clip assembly 1000, or any component thereof, to minimize tilt and/or deflection deformations. Stress relief features may reduce the incidences of tilt and deflection deformations by distributing stress. A stress relief feature may be a notch, cutout, and the like, which may help in mitigating stress concentrations and thus, curtail material deformation. In an example, a stress relief feature may be incorporated into the device-side member 109 to allow the device-side member 109 to exhibit a degree of flexibility at one or more locations, such as at or near the contact point 1030, to aid in the alleviation of pressure and reduction of deflection. Such flexibility may allow the device-side member 109 to maneuver or flex around a physical feature like a camera bump, adapting to the contours of a smart device surface. This capacity for adaptive bending may maintain a secure fit and the structural integrity of the angled clip assembly 1000.

The thickness of angled clip assembly 1000, or any component thereof, may be selected to reduce tilt and/or deflection deformations. The thickness of a member of the angled clip assembly 1000 may be used to mitigate deflection and/or tilt, for example, targeting the flex experienced at the device-side angle 1020 and/or the dial-side angle 1010. For example, a member of the angled clip assembly 1000 may have a thickness within a range, for example, between 4.0 mm to 6.5 mm, such that the design of the angled clip assembly 1000 may exert a level of control over the end deflection 1040 to ensure that it remains within a manageable range of 1 mm to 4 mm.

The thickness of top member 113 may be used to reduce flex in device-side angle 1020 and/or dial-side angle 1010 to minimize deflection and/or tilt. The thickness of top member 113 may be between 4.0 mm to 6.5 mm to maintain end deflection 1040 within a range of 1 mm to 6.5 mm. The thickness of top member 113 may be a factor in enhancing the structural rigidity and stability of top member 113. For example, it may directly contribute to minimizing unintentional flexing, which can lead to tilt or deflection, thus promoting a more stable and reliable alignment between the assembled components.

Examples of the thickness of a member of the angled clip assembly 1000, such as the top member 113 are shown in Table 2.

TABLE 2

| | Net | Deflection with | |
|---|---|---|---|
| Change in thickness | Deflection | 1 deg. Inset | Net Deflection |
| (mm) | (mm) | (mm) | Angle in degs. |
| 0 (no change) | 6.8 | 5.1 | 3.1 |
| 4.5 to 5.0 (0.5 change) | 5.8 | 4.1 | 2.5 |
| 5.0 to 5.5 (0.5 change) | 5.2 | 2.5 | 2.2 |
| 5.5 mm to 6.0 (0.5 change) | 4.8 | 3.1 | 1.9 |
| 6.0 to 6.5 (0.5 change) | 4.4 | 2.7 | 1.6 |

The angled clip assembly 1000 may comprise the dial-side angle 1010. The dial-side angle 1010 may be formed as a result of the structural integration of the top member 113 and the dial-side member 111. In an example, the dial-side member 111 may be attached to an end of the top member 113 to form the dial-side angle 1010. The arrangement and attachment of the dial-side member 111 and the top member 113 may ensure that an angle or a range of angles is maintained within the angled clip assembly 1000 when the angled clip assembly 1000 is attached to a smart device. By maintaining an angle or a range of angles, the angled clip assembly 1000 may mitigate deflection potentially caused by the surface of the smart device, such as a camera surface. For example, the dial-side member 111 may be attached to the top member 113 such that the dial-side angle 1010 is 95 degrees when the angled clip assembly 1000 is unloaded, and may be between 90-100 degrees when the angle clip assembly 1000 (e.g., the contact point 1030, the device-side member 109, and/or the dial-side member 111) is under a load.

The angled clip assembly 1000 may be designed as a continuous piece that comprises the dial-side member 111 and the top member 113 as integral components of the angled clip assembly 1000. This unified design may facilitate the maintenance of an angle or range of angles, such as the dial-side angle 1010, within the angled clip assembly 1000 when it is attached to a smart device to reducing tilt and/or deflection. For example, the arrangement of the dial-side member 111 and the top member 113 may ensure the dial-side angle 1010 may be maintained at 95 degrees in an unloaded state and adjust to stay within the range of 90-100 degrees under load conditions (e.g., at the contact point 1030, the device-side member 109, and/or the dial-side member 111), enhancing the clip functionality and adaptability.

The dial-side angle 1010 may be designed to provide an angle or a range of angles to exert a level of control over the end deflection 1040 to ensure that it remains within a manageable range. For example, the dial-side angle 1010 may be between 90-100 degrees so that the end deflection 1040 may be between 1 mm to 4 mm.

The angled clip assembly 1000 may comprise the device-side angle 1020. The device-side angle 1020 may be formed as a result of the structural integration of the top member 113 and the device-side member 109. In an example, the device-side member 109 may be attached to an end of the top member 113 to form the device-side angle 1020. The arrangement and attachment of the device-side member 109 and the top member 113 may ensure that an angle or a range of angles is maintained within the angled clip assembly 1000 when the angled clip assembly 1000 is attached to a smart device. By maintaining an angle or a range of angles, the angled clip assembly 1000 may mitigate deflection potentially caused by the surface of the smart device, such as a camera surface. For example, the device-side member 109 may be attached to the top member 113 such that the device-side angle 1020 is 84 degrees when the angled clip assembly 1000 is unloaded, and may be between 70-100 degrees when angle clip assembly 1000 (e.g., contact point 1030, device-side member 109, and/or dial-side member 111) is under a load.

The angled clip assembly 1000 may be designed as a continuous piece that comprises device-side member 109 and top member 113 as integral components of angled clip assembly 1000. This unified design may facilitate the maintenance of an angle or range of angles, such as device-side angle 1020, within the angled clip assembly 1000 when it is attached to a smart device to reduce tilt and/or deflection. For example, the arrangement of device-side member 109 and top member 113 may ensure the device-side angle 1020 may be maintained at 84 degrees in an unloaded state and may adjust to stay within the range of 70-100 degrees under load conditions (e.g., at the contact point 1030, the device-side member 109, and/or the dial-side member 111), enhancing the clip functionality and adaptability.

The device-side angle 1020 may be designed to provide an angle or a range of angles to exert a level of control over the end deflection 1040 to ensure that it remains within a manageable range. For example, the device-side angle 1020 may be between 70-100 degrees so that the end deflection 1040 may be between 1 mm to 4 mm.

The angled clip assembly 1000 may be designed to include an opening for securing a smart device. The size of this opening may be dependent on the geometry of the angled clip assembly, which may be influenced by the dimensions and configurations of its constituent components or members. For example, the sizes of the top member 113, the dial-side member 111, and the device-side member 109, along with the angles, such as device-side angle 1020 and the dial-side angle 1010, impact the assembly's geometry. Adjustments to the size or shape of these members, or alterations to the angles, may lead to variations in the opening size, affecting how large a smart device is and how securely the smart device can be accommodated. For instance, the dial-side angle 1010 and the device-side angle 1020, may have their range of angles meticulously defined between 80 to 110 degrees and 70 to 100 degrees, respectively, to ensure that the assembly can effectively mitigate deflection and accommodate a variety of smart device sizes.

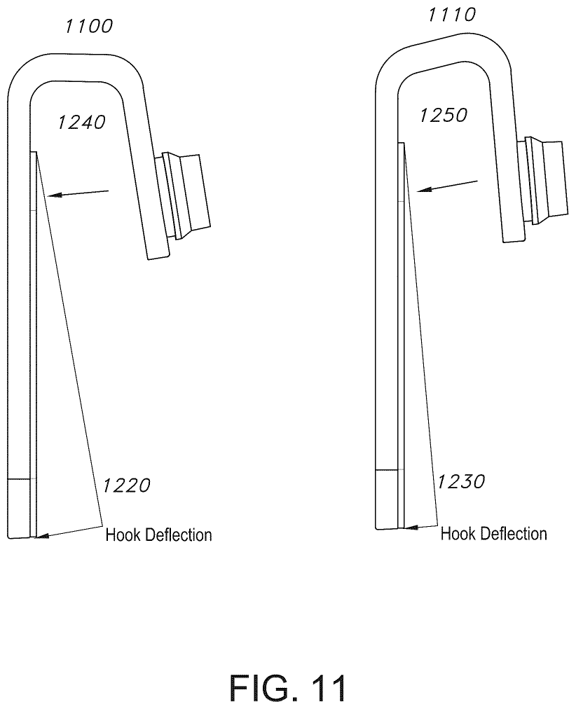

FIG. 11 depicts a side profile view of a first angled clip assembly that comprises a first geometry, and a second angled clip assembly that comprises a second geometry.

The first angled clip assembly 1100 may comprise the dial-side member 1102, the device-side member 1104, the top member 1106, the dial-side angle 1184, and the device-side angle 1182. The first angled clip assembly 1100 may comprise the opening 1122, which may be used for securing a smart device. The geometry of the first angled clip assembly 1100 may be shaped by the dimensions and configurations of its components. For example, a first geometry may be shaped by the sizes of comprise the dial-side member 1102, the device-side member 1104, the top member 1106, the dial-side angle 1184, and the device-side angle 1182.

The first geometry of first angled clip assembly 1100 is determined by the lengths of its components, including the dial-side member 1102, the device-side member 1104, and the top member 1106. Changes to these lengths may influence the first geometry and may affect the size of opening 1122. For example, extending the dial-side member 1102 may increase the opening height 1120, allowing it to accommodate taller devices. Similarly, lengthening the top member 1106 may enlarge the opening width 1150, making it suitable for wider devices. These adjustments illustrate how the assembly's geometry, and thus its device compatibility, may be fine-tuned by modifying component dimensions.

The geometry of the first angled clip assembly 1100 and the size of the opening 1122 may be influenced by the dial-side angle 1184 and the device-side angle 1182. Modifying these angles may alter the assembly's geometry; for example, increasing either angle may enlarge the opening width 1150, enhancing compatibility with wider devices. Modifying these angles beyond a range may compromise the assembly's structural integrity, potentially reducing its capacity to prevent tilt and/or deflection. Component sizes and angles may offer flexibility in device accommodation while considering a balance with the assembly's overall stability and performance.

The dial-side angle 1184 of the first angled clip assembly 1100 may be between 85 to 90 degrees. For example, the dial-side angle 1184 may be 87.5 degrees. The device-side angle 1182 may be between 90 and 100 degrees. For example, the device-side angle 1182 may be 92.5 degrees. These angles may shape the assembly's geometry, influencing both the structural stability and the size of the opening 1122 to ensure compatibility and functionality with a wide array of smart devices.

An inset angle within the context of an angled clip assembly may serve as an element designed to counteract deflection stress, such as those that may arise from direct interaction with a smart device's surface, such as a camera bump. The inset angle may play a role in adjusting the assembly's structural posture to distribute and mitigate pressures, preventing unwanted tilt or deflection of the assembly components under load.

The inset angle within the angled clip assembly is formed between the dial-side member and the device-side member, serving as a measure of their relative orientation. An inset angle of 0 degrees may signify that the dial-side and device-side members are parallel or near parallel to each other, implying a neutral orientation with no convergence at their distal ends. A positive inset angle indicates that the distal ends of both members are angled towards each other, creating a converging orientation. A negative inset angle indicates that the distal ends of both members are angled towards each other, creating a converging orientation. This arrangement facilitates a tailored fit around the smart device and contributes to the assembly's overall stability by optimizing the distribution of forces and enhancing its ability to counteract potential tilt or deflection.

The inset angle 1116 may be formed between the dial-side member 1102 and the device-side member 1104, with the inset angle 1116 potentially ranging from −1 to 1 degree. For example, the inset angle 1116 may be set at 0 degrees, indicating that the dial-side member 1102 and the device-side member 1104 may be parallel or near parallel to each other. This orientation may indicate there is minimal inward or outward tilt at their distal ends.

Regarding the structural components of the first angled clip assembly 1100, top member 1106 may be of top member thickness 1160, which is between 4 mm and 6 mm. For example, top member thickness 1160 may be a thickness of 4.5 mm. Top member thickness 1160 may contribute to ensuring that the assembly may adequately prevent tilt and deflection, thereby preserving the secure attachment of smart devices.

The dimensions of the opening 1122 may be influenced by the first geometry of first angled clip assembly 1100. The opening 1122 may comprise the opening height 1120 and the opening width 1150. The opening height 1120 and the opening width 1150 may be designed to be compatible with various smart device by accounting for the center of smart device camera lens, which may be located between 10 to 40 mm (e.g., 39 mm) from the top of the phone. The opening height 1120 may be within a range of 28 mm to 34 mm. For example, the opening height 1120 may be 31.5 mm to accommodate the distance between the top of the smart device and the center of its camera lens. The opening width 1150 may be between 10 mm to 13 mm. For example, opening width 1150 may be 12 mm to accommodate different smart device widths.

The second angled clip assembly 1110 may comprise the dial-side member 1142, the device-side member 1144, the top member 1115, the dial-side angle 1194, and the device-side angle 1192. The second angled clip assembly 1110 may comprise the opening 1124, which may be used for securing a smart device. The geometry of the second angled clip assembly 1110 may be shaped by the dimensions and configurations of its components. For example, the second geometry may be shaped by the sizes of comprise the dial-side member 1142, the device-side member 1144, the top member 1115, the dial-side angle 1194, and the device-side angle 1192.

The second geometry of the second angled clip assembly 1110 is determined by the lengths of its components, including the dial-side member 1142, the device-side member 1144, and the top member 1115. Changes to these lengths may influence the second geometry and may affect the size of the opening 1124. For example, extending the dial-side member 1142 may increase the opening height 1140, allowing it to accommodate taller devices. Lengthening the top member 1115 may enlarge the opening width 1155, making it suitable for wider devices. These adjustments illustrate how the assembly's geometry, and its device compatibility, may be tuned by modifying component dimensions.

The geometry of the second angled clip assembly 1110 and the size of opening 1124 may be influenced by the dial-side angle 1194 and the device-side angle 1192. Modifying these angles may alter the assembly's geometry; for example, increasing either angle may enlarge the opening width 1155, enhancing compatibility with wider devices. Modifying these angles beyond a range may compromise the assembly's structural integrity, potentially reducing its capacity to prevent tilt and/or deflection. Component sizes and angles may offer flexibility in device accommodation while considering a balance with the assembly's overall stability and performance.

The dial-side angle 1194 of the second angled clip assembly 1110 may be between 90 to 100 degrees. For example, the dial-side angle 1194 may be 95 degrees. The device-side angle 1192 may be between 80 and 90 degrees. For example, the device-side angle 84 may be degrees. These angles may shape the assembly's geometry, influencing both the structural stability and the size of the opening 1122 to ensure compatibility and functionality with a wide array of smart devices.

The inset angle 1118 may be formed between the dial-side member 1142 and the device-side member 1144, with the inset angle 1118 potentially ranging from −2 to 2 degrees. For example, the inset angle 1118 may be set to 1 degree, indicating that the dial-side member 1142 and the device-side member 1144 may be angled towards each other, creating a converging orientation.

Top member 1115 may be of the top member thickness 1170, which is between 4 mm and 6 mm. For example, the top member thickness 1170 may be a thickness of 5.5 mm. The top member thickness 1170 may contribute to ensuring that the assembly may adequately prevent tilt and deflection, thereby preserving the secure attachment of smart devices.

The dimensions of the opening 1122 may be influenced by the second geometry of the second angled clip assembly 1110. The opening 1122 may comprise an opening height 1140 and an opening width 1155. The opening height 1140 and the opening width 1155 may be designed to be compatible with various smart device by accounting for the center of smart device camera lens, which may be located between 10 to 40 mm (e.g., 39 mm) from the top of the phone. The opening height 1140 may be within a range of 28 mm to 38 mm. For example, the opening height 1140 may be 39.15 mm to accommodate the distance between the top of the smart device and the center of its camera lens. The opening width 1155 may be between 10 mm to 13 mm. For example, the opening width 1155 may be 15.35 mm to accommodate different smart device widths.

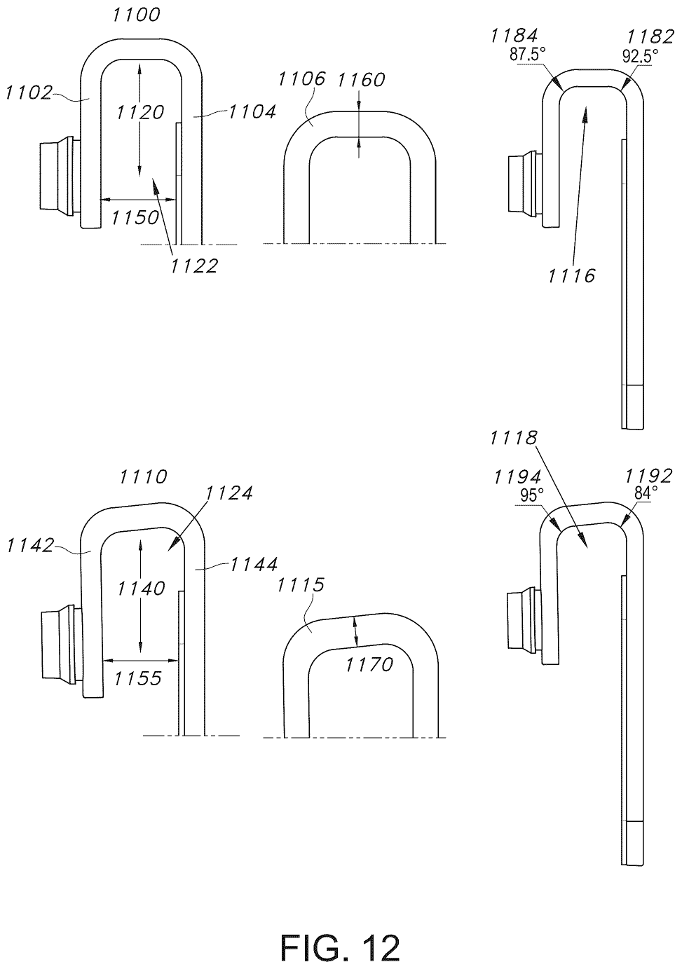

FIG. 12 depicts a side profile view of a first angled clip assembly under a load, and a second angled clip assembly under a load. The first angled clip assembly is first angled clip assembly 1100 and the second angled clip assembly is second angled clip assembly 1110 as described herein. The load may be an attachment to a smart device, which is not shown in FIG. 12 for purposes of simplicity.

The second angled clip assembly 1110 may provide improved performance when compared to the first angled clip assembly 1110. For example, the geometry of the second angled clip assembly 1110 may have a better ability to resist and/or prevent tilt and deflection deformations when compared to the geometry of first angled clip assembly 1100.

The second angled clip assembly 1110 may provide enhanced performance in reducing tilt deformation when compared to the first angled clip assembly 1100. This enhancement may be attributed to the design and geometrical adjustments of the second angled clip assembly 1110, which mitigates tilt by distributing forces more evenly across attachment points. An improvement between designs may be observed in the comparative angles of tilt. For example, the angle of tilt experienced at the location 1250 of the second angled clip assembly 1110 is less than the angle of tilt at location 1240 with the first angled clip assembly 1100. The reduced tilt angle may provide a more stable and secure connection and may lessen the stress imparted on the clip assembly and the attached smart device.

The second angled clip assembly 1110 may provide enhanced performance in reducing deflection deformation when compared to the first angled clip assembly 1100. This enhancement may be attributed to the design and geometrical adjustments of the second angled clip assembly 1110, which mitigate deflection by distributing forces more evenly across various attachment points. An improvement between designs may be observed in the end deflection measurements, where the end deflection 1230 for the second angled clip assembly is smaller (e.g., lower) than the end deflection 1220 for the first angled clip assembly 1100. This reduction in end deflection may ensure a more stable and secure connection and may lower the stress placed on the clip assembly and the attached smart device.

The second angled clip assembly 1110 may provide increased compatibility with smart devices, enabling it to attach to a larger range of smart device sizes or formats. For example, the second angled clip assembly 1110 may provide better compatibility with smart devices than the first angled clip assembly 1100. In an example, the enhanced compatibility provided by the second angled clip assembly 1110 may be due to the assembly's design or geometry, which may feature a larger opening that may accommodate a wider variety of smart device dimensions. The opening (e.g., the opening 1124, shown in FIG. 11) may allow for a more diverse range of devices to be securely attached while managing tilt and deflection deformation, ensuring a more secure and stable connection.

The second angled clip assembly 1110 may provide enhancements over the first angled clip assembly 1100 in smart device compatibility, deflection deformation minimization, and its adeptness in handling surface irregularities like camera bumps. This may be due to the geometry created by one or more angles of the second angled clip assembly 1110 such as the inset angle 1118, the dial-side angle 1194, and the device-side angle 1192.

The inset angle 1118 of the second may be set to 1 degree, which may allow the distal ends of the dial-side and device-side members to be oriented toward each other, creating a converging shape. The converging shape may ensure a more precise and firmer fit against smart devices compared to the first assembly's inset angle 1116, which stands at 0 degrees. For example, the inset angle 1118 may provide better tilt and deflection deformation than the inset angle 1116.

The dial-side angle and device-side angle of the second angled clip assembly 1110 may be selected to provide enhanced tilt and deflection deformation resistance than those of the first angled clip assembly 1100. The dial-side angle 1194 may be set to 95, which may be larger than the dial-side angle 1184 of 87.5 degrees. This increased angle may broaden the opening of the second angled clip assembly 1110, promoting compatibility with a wider array of device sizes and forms while minimizing tilt and deflection deformation (e.g., improving its handling of camera bumps). The device-side angle 1192 may be set to 84, which may be smaller than the device-side angle 1182 of 92.5. This decreased angle may tighten the fit against the device side to promote compatibility with a wider array of devices sizes and forms while minimizing tilt and deflection deformation (e.g., improving its handling of camera bumps).

The geometry of the second angled clip assembly may create a functional tilt in the top member 1115. The end of top member 1115 that connects to the dial-side member 1142 may be designed to be lower or angled downward in comparison to its other end that attaches to the device-side member 1144. This tilt may aid in a better engagement with smart devices, accommodating variations, such as camera bumps, to reduce tilt and deflection deformation.

The thickness of the top member of the second angled clip assembly 1110 may be selected to provide enhanced tilt and deflection deformation resistance than that of the first angled clip assembly 1100. The top member thickness 1260 may be 4.5 mm. The top member thickness 1270 may be 5.5 mm. The top member thickness 1270 may provide enhanced structural rigidity and stability, offering a more substantial resistance against bending forces (e.g., preventing flex in one or more angles). This augmentation in thickness may contribute to a reduction in the end deflection experienced by the assembly. For example, the increased thickness may make the second angled clip assembly 1110 less prone to flexing under load, minimizing both tilt and deflection deformations.

The examples provided herein illustrate that a variety of geometric designs may be utilized for the angled clip assembly, such as shown in Table 3. Table 3 provides different configurations and their potential effects on the end deflection of a clip assembly (e.g., angled clip assembly). Within Table 3, examples 2 through 6 may derived from the example 1. For example, example 1 may be a first design, and examples 2 through 6 may represent a modification of the first design, demonstrating how various geometric modifications may influence the performance of the clip assembly.

TABLE 3

| | Device | | | | | | | | |
|---|---|---|---|---|---|---|---|---|---|
| Example | internal | Height | Width | Dial-Side | Device-Side | Device- | Top | Dial-Side | End |
| Number | filet | increment | Increment | Angle | Angle | Side | Thickness | Thickness | Deflection |
| 1 | 4 | 0 | 0 | 92.5 | 87.5 | 4.5 | 4.5 | 4.5 | 3.0 |
| 2 | 4 | 7.65 | 3.35 | 80 | 100 | 4.5 | 4.5 | 4.5 | 6.4 |
| 3 | 4 | 7.65 | 3.35 | 80 | 100 | 4.5 | 6.5 | 4.5 | 4.1 |
| 4 | 4 | 7.65 | 3.35 | 80 | 100 | 4.5 | 7.5 | 4.5 | 3.7 |
| 5 | 5 | 7.65 | 3.35 | 80 | 100 | 5.5 | 6.5 | 4.5 | 3.3 |
| 6 | 5 | 7.65 | 3.35 | 80 | 100 | 5.5 | 6.5 | 5.5 | 2.8 |

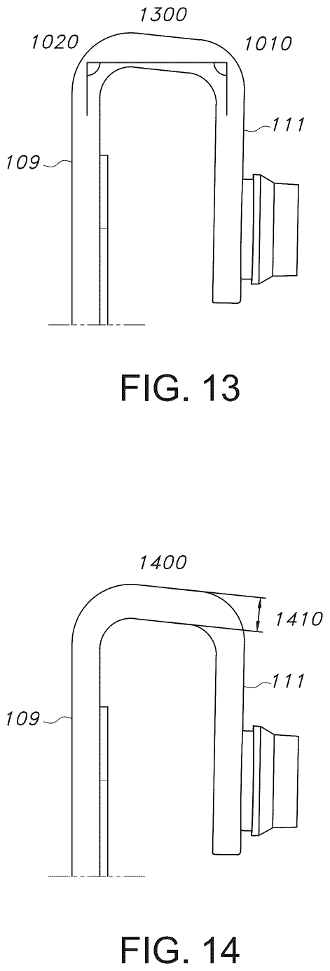

FIG. 13 depicts a side profile view of an angled clip assembly that comprises a geometry with a device side angle and a dial side angle. At 1300, a close up view of the angled clip assembly 1000 may be seen. The device-side angle 1020 and the dial-side angle 1010 may be selected to enhance the stability and attachment efficacy of the assembly, for example, to minimize tilt and deflection deformations. As disclosed herein, the device-side angle and the dial-side angle may be within a range of degrees to allow a clip assembly (e.g., an angled clip assembly) to compensate for one or more surfaces of a smart to distribute stress more evenly across one or more contact points. The stress distribution mechanism may assist the clip assembly in preventing tilt or deflection deformations under load so that the clip assembly may maintain its intended position with minimal deformation. The selection of these angles may assist in optimizing the interaction between the clip assembly geometry and the smart device, allowing for a stable grip that resists the forces that may induce tilt and deflection.

The device-side angle 1020 may be an angle between 70 to 100 degrees. The dial-side angle 1010 may be an angle between 80 to 100 degrees. Table 4 shows examples of angles that may be selected for the dial-side angle 1010 and the device-side angle 1020 as well as their potential effect on deflection.

TABLE 4

| | Dial-Side Angle | Device-Side Angle | Deflection |
|---|---|---|---|
| Example | (degs.) | (degs.) | (mm) |
| 1 | 85 | 95 | 7.3 |
| 2 | 89.5 | 92.5 | 7.2 |
| 3 | 95 | 85 | 6.8 |
| 4 | 100 | 80 | 7.0 |
| 5 | 105 | 75 | 7.2 |

FIG. 14 depicts a side profile view of an angled clip assembly that comprises a top member that may reduce tilt and/or deflection deformation. At 1400, a close up view of the angled clip assembly 1000 may be seen. The thickness of a clip assembly, such as the angled clip assembly 1000, may be selected to reduce tilt and/or deflection deformations. For example, the top member thickness 1410 may be used to mitigate deflection and/or tilt by targeting the flex experienced at the device-side angle and/or dial-side angle. As disclosed herein, the top member thickness 1410 may be within a range, for example, between 4.0 mm to 6.5 mm, such that the design of angled clip assembly 1000 may exert a level of control over the end deflection 1040 to ensure that it remains within a manageable range of 1 mm to 4 mm.

Examples of the thickness for the top member thickness 1410 are described herein, with some examples being shown in Table 5 along with potential effects on deflection.

TABLE 2

| Example | Top Member Thickness (mm) | Deflection (mm) |
|---|---|---|
| 1 | 4.5 | 7.2 |
| 2 | 5.5 | 5.1 |
| 3 | 6.5 | 4.3 |

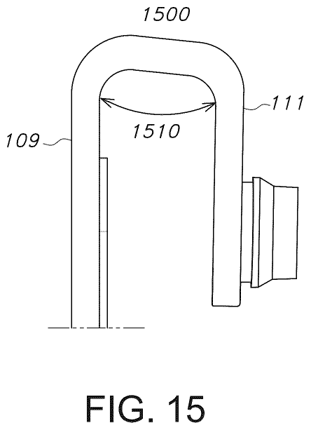

FIG. 15 depicts a side profile view of an angled clip assembly that comprises an internal angle that may reduce tilt and/or deflection deformation. At 1500, a close up view of angled clip assembly 1000 may be seen. The internal angle 1510 may serve as an element designed to counteract deflection stress, such as those that may arise from direct interaction with a smart device's surface, such as a camera bump. The internal angle 1510 may play a role in adjusting the assembly's structural posture to distribute and mitigate pressures, preventing unwanted tilt or deflection of the assembly components under load. As used herein, an internal angle may be referred to as an inset angle.

The internal angle 1510 within the angled clip assembly is formed between the dial-side member and the device-side member, serving as a measure of their relative orientation. An internal angle of 0 degrees may signify that the dial-side and device-side members are parallel or near parallel to each other, implying a neutral orientation with no convergence at their distal ends. A positive internal angle indicates that the distal ends of both members are angled towards each other, creating a converging orientation. A negative internal angle indicates that the distal ends of both members are angled towards each other, creating a converging orientation. This arrangement facilitates a tailored fit around the smart device and contributes to the assembly's overall stability by optimizing the distribution of forces and enhancing its ability to counteract potential tilt or deflection.

The internal angle 1510 may be formed between the dial-side member 111 and the device-side member 109, with the internal angle 1510 potentially ranging from −2 to 2 degree. In an example, the internal angle 1510 may be set at 0 degrees, indicating that the dial-side member 111 and the device-side member 109 may be parallel or near parallel to each other. This orientation may indicate there is minimal inward or outward tilt at their distal ends. In an example, the internal angle 1510 may be set to 1 degree, indicating that the dial-side member 111 and the device-side member 109 may be angled towards each other, creating a converging orientation.

Examples of angles for the internal angle 1510 are described herein, with some examples being shown in Table 6 along with potential effects on deflection.

TABLE 6

| | | | Top | |
|---|---|---|---|---|
| | | Internal | Member | Net |
| | | Angle | Thickness | Deflection |
| Example | Material | (degs.) | (mm) | (mm) |
| 1 | PC-ABS | 1 | 4.5 | 4.9 |
| 2 | PC-ABS | 2 | 4.5 | 3.3 |
| 3 | PC-ABS | 1 | 6.5 | 2.6 |
| 4 | PC-ABS | 2 | 6.5 | 0.9 |
| 5 | PC-ABS Glass Filled | 1 | 4.5 | 1.0 |
| 6 | PC-ABS Glass Filled | 2 | 4.5 | −0.6 |

This application may refer to “determining” various pieces of information. Determining the information can include one or more of, for example, estimating the information, calculating the information, predicting the information, or retrieving the information from memory.

Additionally, this application may refer to “receiving” various pieces of information. Receiving is, as with “accessing”, intended to be a broad term. Receiving the information can include one or more of, for example, accessing the information, or retrieving the information (for example, from memory). Further, “receiving” is typically involved, in one way or another, during operations such as, for example, storing the information, processing the information, transmitting the information, moving the information, copying the information, erasing the information, calculating the information, determining the information, predicting the information, or estimating the information.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as is clear to one of ordinary skill in this and related arts, for as many items as are listed.

We describe a number of examples. Features of these examples can be provided alone or in any combination, across various claim categories and types. Further, embodiments can include one or more of the following features, devices, or aspects, alone or in any combination, across various claim categories and types.

1. A device for attaching a movable optical element to a smart device, the device comprising:
a main body, wherein the main body comprises a first member attached to a second member and a third member such that an inset angle is formed between the second member and the third member, wherein the third member comprises an aperture;
a first contact surface attached to the second member and configured to contact a first smart device surface;
a piston that comprises a second contact surface that is configured to contact a second smart device surface; and
a threaded knob connected to the piston through the aperture, wherein the threaded knob is configured to move the piston in a direction relative to the first surface when turned.

2. The device of claim 1, wherein the inset angle is between −2 to 2 degrees.

3. The device of claim 1, wherein the inset angle is adapted to compensate for a deflection of the second member under a load caused by the first smart device surface.

4. The device of claim 1, wherein the first smart device surface is a camera surface, and wherein the inset angle is adapted to compensate for a deflection of the second member under a load caused by the camera surface.

5. The device for claim 1, wherein the main body is comprised of a resilient material such that the inset angle can increase when a load is applied to the first contact surface.

6. The device for claim 1, wherein the main body comprises at least one of Polycarbonate-Acrylonitrile Butadiene Styrene (PC ABS) or glass-filled PC ABS (PC ABS G).

7. The device of claim 1, wherein the second member is adapted to bend away from the third member when a first load is applied to the first contact surface.

8. The device of claim 7, wherein the third member is adapted to bend away from the second member when a second load is applied to the second contact surface.

9. The device of claim 1, wherein the first member comprises a thickness between 3 mm to 6 mm and is adapted to reduce flex where the first member is attached to the second member and the third member.

10. The device of claim 1, wherein the first member being attached to the second member and the third member comprises a first end of the first member being attached to a proximal end of the second member such that a first angle is formed between the first member and the second member, and a second end of the first member being attached to a proximal end of the third member such that a second angle is formed between the first member and the third member.

11. The device of claim 10, wherein the first angle is between 70 to 100 degrees.

12. The device of claim 10, wherein the second angle is between 80 to 110 degrees.

13. A device for attaching a moveable optical element to a smart device, the device comprising:
a first member, comprising a first end and a second end;
a second member, wherein a proximal end of the second member is attached to the first end to form a first angle;
a third member that comprises a first aperture, wherein a proximal end of the second member is attached to the second end to form a second angle;
a piston that comprises, a shaft, a first surface, and a second surface, wherein the first surface is configured to contact a smart device surface, and wherein the second surface comprises a second aperture for aligning a viewing portion of the moveable optical element with a camera lens of the smart device in a first axis; and
a knob connected to the shaft through the first aperture.

14. The device of claim 13, wherein the first angle is between 70 to 100 degrees.

15. The device of claim 13, wherein the second angle is between 80 to 110 degrees.

16. The device of claim 13, wherein the device further comprises a third angle that is formed between the second member and the third member, and wherein the third angle is between −2 to 2 degrees.

17. The device of claim 16, wherein the first smart device surface is a camera surface, and wherein at least one of the first angle, the second angle, or the third angle is adopted to compensate for a deflection of the second member under a load caused by the camera surface.

18. The device of claim 13, wherein the second member is adapted to bend away from the third member when a first load is applied to the first contact surface.

19. The device of claim 13, wherein the third member is adapted to bend away from the second member when a second load is applied to the second contact surface.

20. The device of claim 13, wherein the first member comprises a thickness between 3 mm to 6 mm and is adapted to reduce flex where at least one of the proximal end of the second member is attached to the first end, or the proximal end of the third member is attached to the second end.

21. A device for attaching an otoscope to a smart device, the device comprising:
a first member;
a second member that comprises an engagement member, wherein the engagement member is configured to engage with a movable otoscope in a first position or a second position, and wherein the second member is attached to the first member to form a first angle;
a third member that comprises an aperture, wherein the third member is attached to the first member to form a second angle;
a piston that comprises a shaft and a contact surface, wherein the contact surface is configured to contact a smart device surface; and
a knob connected to the shaft through the aperture.

22. The device of claim 21, wherein the first angle is between 70 to 100 degrees.

23. The device of claim 21, wherein the second angle is between 80 to 110 degrees.

24. The device of claim 21, wherein the device further comprises a third angle that is formed between the second member and the third member, and wherein the third angle is between −2 to 2 degrees.

25. The device of claim 24, wherein the smart device surface is a camera surface, and wherein at least one of the first angle, the second angle, or the third angle is adopted to compensate for a deflection of the second member under a load caused by the camera surface.

26. The device of claim 21, wherein the second member is adapted to bend away from the third member when a first load is applied to the contact surface.

27. The device of claim 21, wherein the third member is adapted to bend away from the second member when a second load is applied to the contact surface.

28. The device of claim 21, wherein the first member comprises a thickness between 3 mm to 6 mm and is adapted to reduce flex where at least one of the second member is attached to the first member or the third member is attached to the first member.
